(19) United States
(12) Patent Application Publication
(10) Pub. No.: US 2024/0216459 A1
(43) Pub. Date: Jul. 4, 2024

(54) COMPOSITION FOR PREVENTING AND/OR AMELIORATING MENSTRUAL CYCLE SYMPTOMS AND/OR DISCOMFORTS, AND METHODS OF PRODUCING AND USING THE SAME

(71) Applicant: ACCESS BUSINESS GROUP INTERNATIONAL LLC, Ada, MI (US)

(72) Inventors: Mary MURRAY, Irvine, CA (US); Gigi KWOK-HINSLEY, Redondo Beach, CA (US); Yingqin LI, Murrieta, CA (US); Gloria HSU, Orange, CA (US); Arun RAJGOPAL, Grand Rapids, MI (US)

(21) Appl. No.: 18/397,580

(22) Filed: Dec. 27, 2023

Related U.S. Application Data
(60) Provisional application No. 63/435,663, filed on Dec. 28, 2022.

(51) Int. Cl. A61K 36/53; A23L 2/395; A61K 9/00; A61K 36/8965; A61P 15/00

(52) U.S. Cl. CPC A61K 36/53; A23L 2/395; A61K 9/0053; A61K 9/0095; A61K 36/8965; A61P 15/00

(57) ABSTRACT

The described invention relates to a composition for preventing, treating, or relieving menstrual symptoms or discomforts and providing mood improvement in a female patient comprising: (i) an ingredient or an extract of chasteberry fruit; (ii) an ingredient or an extract of saffron red sargol stigma, wherein the ingredient or the extract of saffron red sargol stigma is encapsulated by maltodextrin; and (iii) an excipient. The described invention also relates to a method of using the composition for prevention and amelioration of common menstrual cycle symptoms and/or discomforts in female subjects.

### RELATED APPLICATIONS

The present patent document claims the benefit of the filing date under 35 U.S.C. § 119(e) of Provisional U.S. Patent Application Ser. No. 63/435,663, filed Dec. 28, 2022, which is hereby incorporated by reference.

### BACKGROUND

A large number of symptoms and/or discomforts have been associated with different phases of female menstrual cycle and can be divided into physical, behavioral, and emotional symptoms and/or discomforts. The symptoms and/or discomforts include, e.g., dysmenorrhea and other menstrual irregularities. Physical symptoms and/or discomforts include bloating, abdominal and back cramps and discomfort, change in appetite, weight gain, breast tenderness and pain, acne and headache. Behavioral and emotional symptoms and/or discomforts include anxiety, depression, lethargy, hypersomnia or insomnia, moodiness, irritability, anger, and social withdrawal. Premenstrual syndrome (PMS) also occurs during the female menstrual cycle. PMS is a combination of physical and emotional symptoms that many women get after ovulation and before the start of their menstrual period. The symptoms and/or discomforts associated with PMS include abdominal and muscle discomfort, mood swings, tender breasts, food cravings, fatigue, irritability and depression. It is estimated that as many as 3 of every 4 menstruating women have experienced some form of premenstrual syndrome. The symptoms and/or discomforts associated with the menstrual cycle can vary in intensity from mild to severe and can affect up to 90% of the women. About 5% of North American women suffer from moderate to severe symptoms affecting their daily life activities.

Pharmacologically active agents have been used to treat some symptoms and/or discomforts affecting females during their menstrual cycle. For example, over-the-counter pain relievers (e.g., Ibuprofen, Naproxen, and Aspirin) can be used to help lessen physical symptoms, such as cramps, headaches, backaches, and breast tenderness. Prescription medicines have also been used if over-the-counter pain medicines do not work. For example, hormonal birth control has been used to help with the physical symptoms of PMS, but it may make other symptoms worse (Menstrual Cycle, 2017). Often, a patient may need to try several different types of birth control before she finds one that helps the symptoms. Antidepressants have been used to help relieve emotional symptoms of PMS for some women when other medicines do not help. Selective serotonin reuptake inhibitors, or SSRIs, are the most common type of antidepressant used to treat PMS. Diuretics (“water pills”) have also been used to reduce symptoms of bloating and breast tenderness. Anti-anxiety medicine has been used to help reduce feelings of anxiousness.

However, all the medicines have risk. Drug treatments can have many disadvantages including side-effects and cost, and are not always effective.

As an alternative treatment for the symptoms and/or discomforts associated with female menstrual cycle, including PMS, many patients have turned to plant-based treatments and/or homeopathic remedies.

One such example is the extract from the fruit of Vitex agnus-castus (V. agnus-castus), also called vitex, chaste tree (or chastetree), chasteberry, Abraham's balm, lilac chastetree, or monk's pepper (see, for example, Lauritzen et al., Phytomedicine, 4:183 (1997); Russo and Galletti, Acta Hort., 426:105 (1996); and Milewicz, et al., Arneimittelforschung, 43:752 (1993)). Chasteberry is the fruit of the chaste tree. It is native to western Asia and southwestern Europe and is now common in much of the southeastern United States. Chasteberry has been used for more than 2,500 years to treat various conditions. The leaves and fruits of V. agnus-castus have been used in foods, as flavor and spice, and the berries as a replacement for pepper. The fruit has also been recommended as a hormone-like remedy for alleviating menstrual disorders and as antiepileptic, carminative, energizer, sedative, anticonvulsant, tranquilizer, and for treating digestive disorders. Based on ethnomedicine of different nations, this plant is used for relieving menstrual pain, eye diseases, spasmodic dysmenorrhea, insufficient lactation, treatment of acne, snakebites and scorpion sting, stomachache, and also as antispasmodic, anaphrodisiac, and emmenagogue agent (Zhou J., et al., “5-11 Vitexin 6, a novel lignan, induces autophagy and apoptosis by activating the Jun N-terminal kinase pathway,” Anticancer Drugs, 24:928-936 (2013); Tandon S., et al., “Insect growth regulatory activity of Vitex trifolia and Vitex agnus-castus essential oils against Spilosoma obliqua,” Fitoterapia, 79:283-6 (2008)).

The use of an extract of saffron stigmas has been proposed for prevention of mood disorders related to depression. Saffron is an herbal product used as antispasmodic, sedative, aiding digestion, carminative, diaphoretic, mucokinetic, stimulating sexual desire, soothing pain, easing menstruation, exhilarating, and dispelling gloom (H. Fukui, et al., “Psychological and neuroendocrinological effects of odor of saffron (Crocus sativus),” Phytomedicine, 18(8):726-730 (2011); H. Hosseinzadeh, et al., “Antidepressant effect of Crocus sativus L. stigma extracts and their constituents, crocin and safranal, in mice,” Int. Symp. Saffron Biol. Biotechnol., 650 (2003)). In addition, experimental studies and clinical trials demonstrated that saffron was effective in the treatment of mild to moderate depression (Fukui et al. supra; A. Akhondzadeh Basti, et al., “Comparison of petal of Crocus sativus L. and fluoxetine in the treatment of depressed outpatients: a pilot double-blind randomized trial,” Prog. Neuropsychopharmacol. Biol. Psychiatry, 31(2):439-442 (2007); S. Akhondzadeh, et al., “Comparison of Crocus sativus L. and imipramine in the treatment of mild to moderate depression: a pilot double-blind randomized trial [ISRCTN45683816],” BMC Complement. Altern. Med., 4(1):12 (2004); S. Akhondzadeh, et al., “Crocus sativus L in the treatment of mild to moderate depression: a double-blind, randomized and placebo-controlled trial,” Phytother. Res., 19(2):148-151 (2005); A. Noorbala, et al., “Hydro-alcoholic extract of Crocus sativus L. versus fluoxetine in the treatment of mild to moderate depression: a double-blind, randomized pilot trial,” J. Ethnopharmacol., 97(2):281-284 (2005)). The safranal and crocin of saffron were found to have antidepressant effects. It is likely that crocin exerts its antidepressant effects by inhibiting norepinephrine and dopamine reuptake. Safranal does the same through inhibiting serotonin reuptake (Hosseinzadeh et al. supra; M. Schmidt, et al., “Saffron in phytotherapy: pharmacology and clinical uses,” Wien. Med. Wochenschr., 157(13-14):315-319 (2007)).

Nonetheless, there is still a clear need for clean, high quality and effective medicament/product for simultaneous relief of common menstrual cycle physical and emotional/mood discomforts, having a rapid onset of action, preventing and/or alleviating most, if not all symptoms and/or discomforts, with reduced or no side-effects.

### SUMMARY

The present invention relates to a composition comprising an ingredient or an extract of chasteberry and an ingredient or an extract of saffron red sargol stigma for use as an effective medicament for relief of common menstrual symptoms and/or discomforts. The composition is presented as a natural alternative with the aim of preventing, alleviating, ameliorating, reducing or treating symptoms or discomforts associated with the female menstrual cycle, such as, e.g., pain, aches, cramping, bloating, breast tenderness, mood changes, mood disorders, anxiety, and fatigue and avoiding the need for treatment with synthetic medicines that may have multiple contraindications and/or side effects.

A composition for preventing and/or menstrual cycle symptoms or discomforts, comprising: (a) an ingredient or an extract of chasteberry fruit; (b) an ingredient or an extract of saffron red sargol stigma, wherein the ingredient or the extract of saffron red sargol stigma is encapsulated by maltodextrin; and (c) an excipient. The extraction ratio of the extract of chasteberry fruit is 6:1, and the extract of chasteberry fruit is extracted with a 50% ethanol extraction. The extraction ratio of the extract of chasteberry fruit is 3.33:1 standardized to 2% crocins, and the extract of chasteberry fruit is extracted with 30% ethanol. The amount of the extract of chasteberry in the composition is in the range of from about 20 to about 40 mg. The amount of the extract of chasteberry in the composition is about 30 mg. The amount of the extract of saffron red sargol stigma in the composition is in the range of from about 40 to about 50 mg. The composition may further comprise at least one other botanical ingredient selected from the group consisting an ingredient or an extract of ginger, turmeric, gingko biloba, fennel, dong quai (Angelica sinensis), and pine bark. In the described composition, the excipient comprises a disintegrant, a binder, a surfactant, an emulsifier, a viscosity modifier, a lubricant, a sweetener, a pH-adjusting agent, a preservative, a flavoring agent, a coloring agent, or an antioxidant. In the composition, the excipient is selected from gellan gum, carob bean gum, locust bean gum, carrageenan, alginates, agar, guar gum, xanthan gum, carboxymethyl cellulose, clear starch, pectin, gelatin, cornstarch, katakuri starch, potato starch, and gum arabic. The excipient may comprise a sweetener selected from high fructose corn syrup, mannose, maltose, glucose polymers, sucrose, glucose, dextrose, lactose, galactose, fructose, polysaccharides, rice syrup, honey, saccharin, cyclamates, acetosulfam, sorbitol, sucralose, xylitol, erythritol, Stevia extract, L-aspartyl-L-phenyl-alanine ester, L-aspartyl-D-alanine alkyl amides, L-aspartyl-L-1-hydroxymethylalkaneamide, and L-aspartyl-1-hydroxyethylalkaneamide. The excipient may comprise a pH-adjusting agent selected from hydrochloric acid, citric acid, sodium hydrogen carbonate, potassium hydroxide, sodium hydroxide, and sodium carbonate. The excipient may comprise a preservative selected from sorbic acid, benzoic acid, sodium benzoate, calcium benzoate, potassium benzoate, potassium sorbate, calcium sorbate, and sodium sorbate. The excipient may comprise a flavoring agent selected from almond oil, amaretto oil, anethole, anise oil, benzaldehyde, blackberry, black walnut oil, blueberry, caraway, caraway oil, cardamom oil, cardamom seed, cherry juice, cherry syrup, cinnamon, cinnamon oil, cinnamon water, citric acid, citric acid syrup, clove oil, cocoa, coriander oil, dextrose, eriodictyon, ethyl acetate, ethyl vanillin, fennel oil, ginger, glucose, glycerin, glycyrrhiza, grape, honey, lavender oil, lemon oil, lime, mannitol, methyl salicylate, myristica oil, orange oil, orange peel, orange syrup, peppermint, peppermint oil, peppermint water, phenylethyl alcohol, pineapple, raspberry juice, raspberry syrup, rosemary oil, rose oil, rose water, sarsaparilla syrup, sorbitol, spearmint, spearmint oil, strawberry, sucrose, thyme oil, tolu balsam, tropical, vanilla, vanillin, and wild cherry syrup. The composition may be a food product. The composition may be a dietary supplement. The composition may be in a form of a beverage or a gel. The composition may be a packaged beverage provided in a unit that contains between 10-1000 mL of the beverage. The composition may be a packaged beverage provided in a unit that contains between 10-500 mL of the beverage. The composition may be in a form of an instant beverage. The composition may be a packaged gel, where the packaged gel may be provided in a unit that contains between 5-100 grams of the gel, or the packaged gel may be provided in a unit that contains between 30-40 grams of the gel. The beverage or the gel can be made by reconstituting a dry powder with an aqueous fluid. The composition may be in a solid dose format. The composition may be for consumption once or more times a day. The composition may be for daily consumption for a minimum of 1 menstrual cycle; the composition may be for daily consumption for a minimum of 2 menstrual cycles; the composition may be for daily consumption for a minimum of 3 menstrual cycles, the composition may be for daily consumption for a minimum of 6 menstrual cycles. The menstrual cycle symptoms or discomforts comprise physical, physiological and/or psychological symptoms or discomforts experienced during the monthly menstrual cycle. The physical menstrual symptoms or discomforts are bloating, abdominal discomfort, lower back pain, cramps in the pelvic area, constipation, diarrhea, swelling or tenderness in the breasts, cyclic acne, joint or muscle pain, and food cravings. The psychological/emotional symptoms or discomforts are stress, anxiety, difficulty with sleep, headache, feeling tired/fatigue, mood swings/fluctuations, increased emotional sensitivity, changes in interest in sex, problems with concentration and memory, and depression.

Another embodiment relates to an instant beverage comprising: (a) an ingredient or an extract of chasteberry fruit; (b) an ingredient or an extract of saffron red sargol stigma, wherein the ingredient or the extract of saffron red sargol stigma is encapsulated by maltodextrin; and (c) an excipient.

Another embodiment relates to a capsule comprising any of the described composition(s).

Another embodiment relates to a method of preventing and/or ameliorating menstrual cycle symptoms and/or discomforts in a subject in need thereof, comprising orally consuming an effective amount of the described composition to prevent and/or ameliorate the subject's menstrual cycle symptoms or discomforts.

Another embodiment relates to a method of preventing and/or ameliorating menstrual cycle symptoms and/or discomforts in a subject in need thereof, comprising orally consuming an effective amount of (a) an extract of chasteberry fruit; (b) an extract of saffron red sargol stigma, wherein the extract of saffron red sargol stigma is encapsulated by maltodextrin, to prevent and/or ameliorate the subject's menstrual cycle symptoms or discomforts. In the method the ingredients (a) and (b) provide prevention and/or amelioration of the menstrual cycle symptoms and/or discomforts, wherein the menstrual cycle symptoms and/or discomforts comprise physical, physiological and/or psychological/emotional symptoms or discomforts experienced during the monthly menstrual cycle of the subject. The physical menstrual symptoms and/or discomforts are bloating, abdominal discomfort, lower back pain, cramps in the pelvic area, constipation, diarrhea, swelling or tenderness in the breasts, cyclic acne, joint or muscle pain, and food cravings. The psychological/emotional symptoms and/or discomforts are stress, anxiety, difficulty with sleep, headache, feeling tired/fatigue, mood swings, increased emotional sensitivity, changes in interest in sex, problems with concentration and memory, and depression. In the method, the ingredients (a) and (b) are orally consumed as a composition. In the method, the step of orally consuming the ingredients (a) and (b) may occur prior to, during, or after the onset of the menstrual cycle symptoms and/or discomforts. In the method, the step of orally consuming the ingredients (a) and (b) may occur once or more times per day. In the method, the step of orally consuming the ingredients (a) and (b) may occur daily for a minimum of 1 menstrual cycle, or daily for a minimum of 2 menstrual cycles, or daily for a minimum of 3 menstrual cycles, or daily for a minimum of 6 menstrual cycles. In the method, the subject may be orally consuming: i) between about 20 to about 40 mg per day of the extract of chasteberry fruit; ii) between about 30 to about 60 mg per day of the extract of saffron red sargol stigma.

Yet another embodiment relates to the use of the described composition for ameliorating of menstrual symptoms and/or discomforts in a subject.

Yet further embodiment relates to the use of the described composition for preventing menstrual symptoms and/or discomforts a subject.

Another embodiment relates to a composition for upregulating the expression of the XIST gene in a female subject comprising: i) between about 20 to about 40 mg per day of the extract of chasteberry fruit; ii) between about 30 to about 60 mg per day of the extract of saffron red sargol stigma.

### BRIEF DESCRIPTION OF THE DRAWINGS

FIG. 1 depicts inflammatory signal pathways of nuclear transcription kappa factor beta leading to the release of eicosanoids from Chen et al., 2015.

FIG. 2 is an illustration from Shenoda et al, 2019.

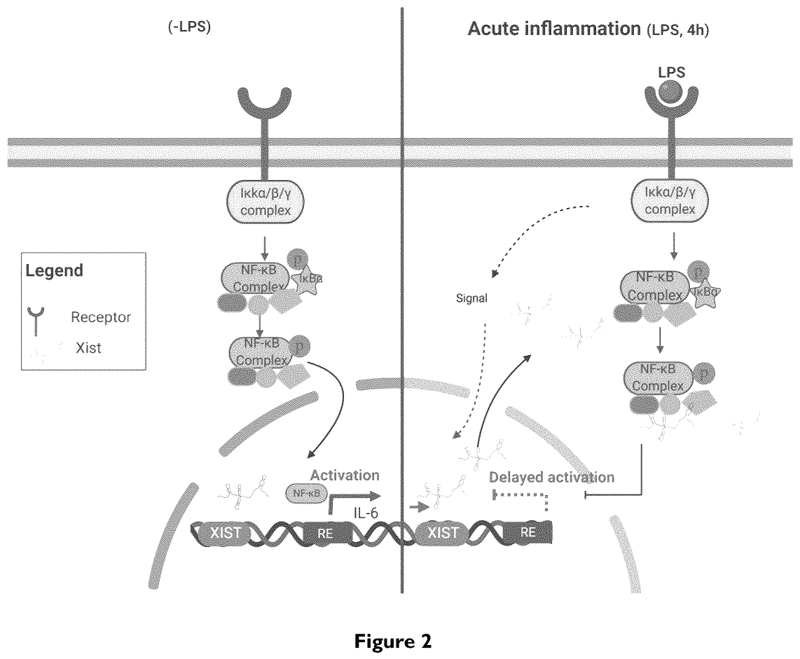

FIG. 3 is a flowchart of steps of the study outlined in Example 3.

### DETAILED DESCRIPTION OF THE PRESENTLY PREFERRED EMBODIMENTS

The present invention will now be described more fully herein after. For the purposes of the following detailed description, it is to be understood that the invention may assume various alternative variations and step sequences, except where expressly specified to the contrary. Thus, before describing the present invention in detail, it is to be understood that this invention is not limited to particularly exemplified embodiments that may of course, vary.

The present invention relates to a composition comprising an ingredient and/or an extract of chasteberry fruit, and an ingredient and/or an extract of saffron red sargol stigma, wherein the ingredient or the extract of saffron red sargol stigma is encapsulated by maltodextrin, for use as an effective medicament for relief of common menstrual cycle symptoms and/or discomforts. Specifically, the ingredients or the composition are for use as effective medicaments for preventing or alleviating the physical, physiological and/or psychological symptoms of female patients associated with the female menstrual cycle.

Unless otherwise defined, all terms technical and scientific used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention.

As used herein, the singular forms “a,” “an,” and “the” include plural reference unless the context clearly dictates otherwise. The term “and/or” means one or all of the listed elements or a combination of any two or more of the listed elements.

The terms “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, “around,” “about” or “approximately” shall generally mean within 20 percent, preferably within 10 percent, and more preferably within 5 percent of a given value or range. Numerical quantities given herein are approximate; meaning that the terms “around,” “about” or “approximately” can be inferred if not expressly stated. When the term “about” is used in describing a value or an endpoint of a range, the disclosure should be understood to include both the specific value and end-point referred to.

As used herein, the terms “comprising,” “including,” “having,” “containing,” “involving,” and the like are to be understood to be open-ended, i.e., to mean including but not limited to.

The term “composition” is used herein to describe a formulation that includes an ingredient and/or extract of chasteberry fruit, and an ingredient and/or an extract of saffron red sargol stigma. The term refers to a comestible formulation that is suitable for oral ingestion by the subject (e.g., the human female subject). Exemplary compositions that include an ingredient and/or extract of chasteberry fruit, and an ingredient and/or an extract of saffron stigmas include, but are not limited to: sprays (e.g., aerosols), powders, chewing gum, ingestible solids, gels, aqueous beverages, dry powder (e.g., a powder that can be directly consumed or that can be reconstituted with liquid to provide a beverage as defined herein), nutritional bars, lozenges, tablets, capsules, wafers, pastes, and the like. Other compositions are described herein. In certain preferred embodiments, the composition described herein is an aqueous beverage.

An “extract” is the material which dissolves in a solvent after contacting a plant substance with a suitable solvent. Preferably, the plant substance is contacted for a sufficiently long period of time and at a suitable temperature so that substantially all of the soluble material is removed by the solvent. The solvent together with the dissolved soluble plant material is referred to as the “liquid extract.” The term “extract” refers to the soluble plant material remaining after removal of the solvent. The solvent can be removed by any suitable means, including evaporation, lyophilization, spray drying and the like.

As used herein the term “an extract of chasteberry fruit” refers to one or more natural materials, in the form of a single material or ingredient (e.g., inorganic, organic, salt, etc.) or a mixture of natural materials obtained from the chasteberry plant (Vitex agnus-castus).

As used herein the term “an extract of saffron red sargol stigma” refers to one or more natural materials, in the form of a single material or ingredient (e.g., inorganic, organic, salt, etc.) or a mixture of natural materials obtained from Crocus sativus L. (Iridaceae), commonly known as “saffron,” which is a perennial stemless herb that is widely cultivated in various countries, such as India and Greece. The saffron used in the described compositions is preferably grown and manufactured in Europe.

As used herein, the expression “active combination” refers to the ability of the combination of the described extracts to exert a protective/preventative/therapeutic effect in the context of improving the physical, physiological and/or psychological symptoms and/or discomforts of females during their menstrual cycle, as disclosed herein. Neither of the components is regarded as an additive, such as carrier, diluent, or excipient.

As used herein, the terms “effective amount” or “pharmaceutically or therapeutically effective amount” refer to the amount of the active ingredient(s), the extract(s), to be administered orally to the subject to trigger the desired effect without or causing minimal toxic adverse effect against the subject, and/or without undesirable side-effects. One skilled in the art should know that the effective amount can vary from one individual to another due to the external factors such as age, sex, diseased state, races, body weight, formulation of the composition, availability of other active ingredients in the formulation, and so on.

The term “excipient” refers to a substance which helps to absorb any of the components of the product of the invention, stabilizes said components or helps in the preparation of the composition. Thus, excipients can have the function of keeping the components bound together, such as for example starches, sugars or celluloses, a sweetening function, a colorant function, the function of protecting the medicament from the external medium, such as for example isolating it from the air and/or moisture, a filler function for a tablet, capsule or any other form of formulation, such as for example di basic calcium phosphate, a disintegrating function to facilitate the dissolution of the components and their absorption in the intestine, without excluding other types of excipients not mentioned in this paragraph. Therefore, the term “excipient” is defined as the substance which, included in dosage forms, is added to the active substances or their associations in order to enable their preparation and stability, modify their organoleptic properties or determine the physical/chemical properties of the pharmaceutical composition and its bioavailability. The “pharmaceutically acceptable” excipient should not interact with the activity of the active compounds of the described composition. Examples of excipients are binding agents, fillers, disintegrants, lubricants, coatings, sweeteners, flavorings and colorants. More specific, non-limiting examples of acceptable excipients are starches, sugars, xylitol, sorbitol, calcium phosphate, steroid fats, talc, silica or glycerin, among others.

By “administering” and “administration” is meant a mode of delivery. A daily dosage can be divided into one, two, three or more doses in a suitable form to be administered one, two, three or more times throughout a time period. In preferred embodiments of the present invention, the described compositions and solutions are prepared for daily oral consumption for a minimum of 1 menstrual cycle; alternatively, daily oral consumption for a minimum of 2 menstrual cycles; alternatively, daily oral consumption for a minimum of 3 menstrual cycles; or, alternatively, daily oral consumption for a minimum of 6 menstrual cycles or longer.

By “preventing” or “reducing the likelihood of” is meant reducing the severity, the frequency, and/or the duration of a condition or disorder (e.g., menstrual symptoms and/or discomforts) or the symptoms thereof. For example, reducing the likelihood of or preventing cramping is synonymous with prophylaxis of cramping.

By “treating” or “ameliorating” or “alleviating” is meant administering a composition for therapeutic purposes or administering treatment to a subject already suffering from a disorder to improve the subject's condition. By “treating a symptom and/or discomfort” or “ameliorating a symptom and/or discomfort” is meant that the a symptom and/or discomfort of a condition or disorder (e.g., menstrual symptom and/or discomfort) and the symptoms associated with the condition or disorder are, e.g., alleviated, reduced, cured, or placed in a state of remission. As compared with an equivalent untreated control, such amelioration or degree of treatment is at least 2%, 5%, 10%, 20%, 30%, 40%, 50%, 60%, 70%, 80%, 90%, 95%, 99%, or 100%, as measured by any standard, suitable technique.

By “beverage” is meant a composition that is not in solid or gas form, such as a liquid or semi-liquid that is designed to enter into the mouth of a subject and be orally consumed or ingested. A beverage may be in a ready-to-drink liquid form (e.g., may be consumed without modification) or in a liquid, solid, or concentrated form (e.g., capsule or stick pack), which can be transformed into a ready-to-drink liquid form with an addition of another liquid (e.g., water), such as an instant beverage.

By “instant beverage” is meant that the described composition is in a form of a premix, which may be a dry powder (e.g., capsule or stick pack) of beverage flavor that can blend with water and other solvents. Instant beverages premixes are available in powder, granules, and paste forms.

As described herein, the term “subject” is equivalent to the terms “individual” and “patient” whereby the terms can be used interchangeably. “Subject” means any animal belonging to any species. Examples of subjects include, but are not limited to, commercially bred animals such as birds (hens, ostriches, chickens, geese, partridges, etc.), rabbits, hares, domestic animals (dogs, cats, etc.), livestock such as sheep and goat livestock, pigs, wild boars, horses, ponies, etc., and cattle (bulls, oxen, etc.). In a particular embodiment, the subject is a mammal, preferably a primate, more preferably a human being of any race, sex or age.

In a particular preferable embodiment, the subject is a human female that is in her fertile period of life and has menstrual cycle. The fertile period of life can vary between individuals. For girls and women in Western countries, the average age for a first period is 12 to 13, but it can start as early as nine and as late as 16, and can last for decades, typically until late 40s, but can also continue into the 50s or 60s for some women. In preferred embodiment, the subject is a human female that is in her fertile period of life and has menstrual cycle and is experiencing menstrual cycle symptoms and/or discomforts.

Specifically, with reference to female menstrual cycle, the subject is female. However, the use of the described composition may not be limited to females. Any individual experiencing similar or the same symptoms to female menstrual cycle discomforts (i.e., symptoms of pain, aches, cramping, bloating, mood changes, anxiety and fatigue) may benefit from the use of the described composition.

The terms, “menstrual cycle” and “menstrual health cycle” can be used synonymously and refer to the cycle that prepares female body for pregnancy. If the subject is not pregnant, the hormones send a signal to the uterus to shed its lining. This becomes the “period” or “menstruation.” Once the period starts, the cycle starts again. A menstrual cycle is measured from the first day of individual's period to the first day of the individual's next period. The average length of a menstrual cycle is 28 to 29 days, but every woman's cycle is different. For example, teenagers might have cycles that last 45 days, whereas women in their 20s to 30s might have cycles that last 21 to 38 days.

### I. Active Components of the Composition

The described invention related to a composition comprising (i) an ingredient or an extract of chasteberry fruit, (ii) an ingredient or an extract of saffron red sargol stigma, wherein the ingredient or the extract of saffron red sargol stigma is encapsulated by maltodextrin, and (iii) an excipient

### Chasteberry

Chasteberry (Vitex agnus-castus), or monk's pepper, is the fruit of the chaste tree. It is native to western Asia and southwestern Europe and

The fruit (i.e., berry) and the leaves of the chaste tree contains a number of active constituents, including flavonoids (e.g., vitexin, casticin, kaempferol, orientin, quercetagetin, and isovitexin), iridoid glycoside (e.g., agnuside and aucubin), p-hydroxybenzoic acid, alkaloids, essential oils (i.e., limonene, cineol, pinene, and sabinene), fatty oils, diterpenoids and steroids. They occur in the fruits and in the leaves.

A single ingredient, a mixture of two or more of these active compounds, or an extract of chasteberry fruit may be employed in the described composition.

Chasteberry can be incorporated in the described composition in many different forms including chasteberry/chasteberry fruit extract, chasteberry/chasteberry fruit powder, one or more active compounds of chasteberry/chasteberry fruit and mixtures thereof. Also, for any specific active compound of chasteberry for which suitable synthesis routes are known, the active compound can be prepared synthetically. Preferably, chasteberry is included in the composition as an extract of chasteberry fruit.

Any appropriate method can be used to prepare chasteberry/chasteberry fruit extract. For example, chasteberry extract can be made by hydrodistillation, steam distillation, leaching, solvent extraction, pressing and extraction with supercritical carbon dioxide.

In a preferable embodiment, the described composition comprises an extract of chasteberry/chasteberry fruit.

In one embodiment, the extract of chasteberry/chasteberry fruit used in the described composition has an extraction ratio of 6:1 made with a 50% ethanol extraction. The vegetable matter/native extract (DER): 10/1.

The extract of chasteberry/chasteberry fruit may be standardized to one or more of compounds such as aucubin, casticin and agnuside.

### Saffron

Crocus sativus L. stigma commonly known as saffron is a perennial stemless herb of the Iridaceae family that is widely cultivated in Iran. Bright red saffron is the stigma of purple saffron flower.

Commercial saffron comprises the dried red stigma with a small portion of the yellowish style attached. Chemical analysis of saffron has shown the presence of more than 150 components in the stigmas of saffron. In addition to the three major components in saffron (crocetin esters, picrocrocin and safranal), it also contains other cartenoids, carbohydrates, raw fiber, proteins, fats, anthocyanins, flavonoids, vitamins (riboflavin and thiamine), minerals, and many other elements which confer nutritional properties and are beneficial to health.

As described herein, the described composition includes saffron. The saffron in the described composition is an ingredient or an extract of saffron stigmas (Crocus sativus L.). The saffron stigma containing the useful components may be conventionally harvested from the pistils of open red or purple saffron flowers. In general, a big saffron bulb weighing about 30 g gives rise to only about 6 flowers, each having three-divided stigma.

Saffron can be incorporated in the described composition in many different forms including saffron extract, saffron powder, one or more active compounds of saffron and mixtures thereof. Also, for any specific active compound of saffron for which suitable synthesis routes are known, the active compound can be prepared synthetically. Preferably, saffron is included in the composition as an extract of saffron stigmas.

Any appropriate method can be used to prepare saffron extract. For example, saffron extract can be made by hydrodistillation, steam distillation, leaching, solvent extraction, pressing and extraction with supercritical carbon dioxide.

In a preferred embodiment, the saffron for use in the described composition is derived from red sargol saffron stigma. Unlike other saffron extracts, the saffron used in this composition is processed by a unique extraction and drying technology to encapsulate the saffron extract by maltodextrin in order to retain saffron active metabolites (e.g., crocins, safranal) and to protect potency and stability of the active components. One example of the extraction and drying technology was previously described in U.S. Patent Publication No. 2019/0160133 A1, which is incorporated herein in its entirety. Specifically, as described in U.S. Patent Publication No, 2019/0160133 A1, after impregnation step, the procedure can also include an emulsion step and/or an encapsulation step for the obtained extract. This step consists of high-speed stirring of the extraction solution containing the bulking agent and, possibly, the auxiliary substance. It can be carried out using auxiliary substances such as acacia gum, cyclodextrins, or fatty substances.

In a preferable embodiment, the extract of saffron is prepared using Techcare™ extraction method as described in U.S. Pat. Pub. No. 2019/0160133 A1, which is incorporated herein in its entirety. Specifically, the extract of saffron can be obtained via a procedure including the implementation of the following steps, completed using saffron as a raw material: (i) possible drying, (ii) grinding, preferentially between 50 and 500 μm, (iii) aqueous or hydroalcoholic extraction, or extraction using an organic solvent, (iv) impregnation of the extract obtained onto a support, and (v) thermal treatment.

The aqueous or hydroalcoholic extraction steps, or extraction using an organic solvent and impregnation of the extract obtained onto a support, constitute the known extraction procedure, named Tech'Care Extraction®.

The thermal treatment step can be carried out at any moment during this procedure, preferentially at the end of the procedure, on the raw material saffron, and completes the Tech'Care Extraction® procedure.

In a preferred embodiment, the thermal treatment step in the implementation of this procedure is a thermal treatment step in a chamber for at least 2 hours, even more preferentially for at least 24 hours at a temperature between 30° C. and 95° C., even more preferentially at a temperature between 30° C. and 60° C.

Grinding can be carried out by any suitable known means, particularly by a granulating mill, a pin mill or a hammer miller, with preference given to a pin mill.

The extraction step can be carried out by any suitable known means. In the case of aqueous extraction, the ground substance is introduced into water at a ratio of 50 g/L. In the case of hydroalcoholic extraction, the solvent can be ethanol, with preference given to ethanol at 60% v/v. The ground substance is introduced into the hydroalcoholic solution at a ratio of 50 g/L.

In the case of extraction using an organic solvent, the solvent can be methanol or ethyl acetate, with preference given to methanol at 30% v/v. The ground substance is introduced into the organic solvent at a ratio of 100 g/L.

After extraction, the procedure can also include an acidification step. This step consists of adding acid into the aqueous or hydroalcoholic solvent. It enables the pH of the extraction solution to be reduced to between 3 and 5. It can be carried out in the following conditions: adding citric acid or hydrochloric acid into the hydroalcoholic solvent to adjust the pH to 4. The step of impregnation onto a support consists of adding a bulking agent into the extraction solution. The support or bulking agent can be selected from among the following components: maltodextrin, sugar, silica, and acacia gum, but maltodextrin is preferred.

As described above, after this impregnation step, the procedure can also include an emulsion step and/or an encapsulation step for the obtained extract. This step consists of high-speed stirring of the extraction solution containing the bulking agent and, possibly, the auxiliary substance. It can be carried out using auxiliary substances such as acacia gum, cyclodextrins, or fatty substances.

In one preferable embodiment, the extraction ratio of saffron red sargol stigma can vary as long as the ingredient or the extract contains 2% crocin. The extraction ratio can range from 5:1 to 3.33:1 made by extraction with 30% ethanol.

### Maltodextrins (MDs)

Maltodextrins (MDs) are a class of carbohydrates (CHOs) extracted from a range of botanical sources. They are industrially produced by enzymatic or acid hydrolysis of the starch, followed by purification and spray drying (Takeiti et al., “Morphological and physicochemical characterization of commercial maltodextrins with different degrees of dextrose-equivalent,” Int. J. Food Propert. 2010:411-425). The resulting commercially available, mostly white, powders are of high purity and microbiological safety and can be used in a wide range of food and beverage products, including baked goods and sports drinks. MDs include digestible and resistant-to-digestion types of MDs. Most MDs are fully soluble in water and exert other important functionalities, such as gelling or freeze control.

MDs can be produced by hydrolysis of starch from different botanical sources. During the production process, native starch is heated in the presence of water, causing the crystalline structure of starch granules to swell and be broken irreversibly. This gelatinization process makes starch available for enzymatic or acidic degradation, or a combination of both. After degradation, chains of D-glucose units are left with varying length and appearance. Digestible MDs ((C₆H₁₀O₅)ₙH₂O) have a relatively short chain length and can be defined as saccharide polymers obtained from edible starch having a so-called dextrose equivalency (DE) of less than 20 (Takeiti et al., supra).

Starch granules mainly contain varying amounts of two types of glucose polymers: amylose and amylopectin, which differ in molecular structure. In amylose, glucose units are linked in a linear structure by α1,4 glycosidic links while some glucose units in amylopectin are linked by α1,6 bonds, resulting in branched structures. Most starches contain approximately 70-80% amylopectin and roughly 20-30% amylose.

In certain preferred embodiments, MDs encapsulate the saffron extract in order to retain saffron active metabolites (e.g., crocins, safranal) and to protect potency and stability of the active components.

### Compositions

In one embodiment, the described composition is a nutraceutical composition, and comprises nutraceutically or pharmaceutically acceptable excipients. The “nutraceutical composition” is the formulation of the set of components that form at least the product of the invention (the composition comprising an ingredient and/or an extract of chasteberry fruit, and an ingredient and/or an extract of saffron stigmas), which has at least one application in improving the physical, physiological and/or psychological state of a subject, which implies a general improvement of their state of health, as well as an increase in their quality of life. In a preferable embodiment, the “nutraceutical composition” is the formulation is a food (e.g., beverage) containing health-giving ingredient and/or extract of chasteberry fruit, and health-giving ingredient and/or extract of saffron stigmas and having medicinal benefit

Another embodiment relates to the use of the composition, as described above, to prepare a medicament or a nutraceutical. The medicament or nutraceutical comprises the composition in an effective dose. As used herein, the term “effective dose” refers to that concentration of the components of interest of the composition, which when administered is capable of producing preventative effect and/or clear relief of common menstrual cycle discomforts (physical, physiological, and/or psychological discomforts) in a subject.

The described composition can include a dose of 5-100 mg per day of an ingredient or an extract of chasteberry fruit; more preferably, 10-80 mg per day of an ingredient or an extract of chasteberry fruit; more preferably, 20-60 mg per day of an ingredient or an extract of chasteberry fruit; more preferably, 20-40 mg per day of an ingredient or an extract of chasteberry fruit; most preferably 30-40 mg per day of an ingredient or an extract of chasteberry fruit. Alternatively, described composition can include a dose 5 mg, 15 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 80 mg, 85 mg, 90 mg, 95 mg, and 100 mg per day of an ingredient or an extract of chasteberry fruit.

The described composition can include a dose of 5-100 mg per day of an ingredient or an extract of saffron stigmas; more preferably, 10-80 mg per day of an ingredient or an extract of saffron stigmas; more preferably, 30-60 mg per day of an ingredient or an extract of saffron stigmas; more preferably, 20-50 mg per day of an ingredient or an extract of saffron stigmas; most preferably 30-50 mg per day of an ingredient or an extract of saffron stigmas; most preferably 40-50 mg per day of an ingredient or an extract of saffron stigmas. Alternatively, described composition can include a dose of 5 mg, 15 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 80 mg, 85 mg, 90 mg, 95 mg, and 100 mg per day of an ingredient or an extract of saffron stigmas.

In one preferred embodiment, a composition comprises 30 mg chasteberry (for a daily dose) at the extract ratio of 6:1 made with a 50% ethanol extraction, and 45 mg saffron red sargol stigma at the extraction ratio of 3.33:1 standardized to 2% crocins and extracted with 30% ethanol. The dose of 45 mg per day is recommended to maintain the same level of crocins per daily dose. The ingredient or the extract of saffron red sargol stigma is encapsulated by maltodextrin.

### II. Additional Active Components of the Composition

In certain embodiments, the described composition may include at least one other active ingredient or an extract, including an ingredient or an extract of ginger, turmeric, gingko biloba, fennel, dong guai, and/or pine bark.

### Ginger

Ginger (Zingiber officinale), also commonly called ginger root, is a flowering plant whose rhizome, ginger root or ginger, is widely used as a spice and a folk medicine. It is a herbaceous perennial which grows annual pseudostems (false stems made of the rolled bases of leaves) about one meter tall bearing narrow leaf blades.

Ginger contains 1-4% essential oil (oleoresin). The active compounds of ginger, which may be employed in the described composition include, but are not limited to, 1,8-cineole, 10-dehydrogingerdione, 10-gingerol, 6-gingerdione, 6-gingerol, shogaols (e.g., [4]-shogaol, [6]-shogaol, [8]-shogaol, [10]-shogaol, and [12]-shogaol), 8-β-17-epoxy-λ-trans-12-ene-15,16-diol, 8-gingerol, 9-oxo-nerolidol, acetaldehyde, acetic acid, alanine, α-linolenic-acid, α-linolenic acid, α-phellandrene, α-piene, α-terpinene, α-terpineol, α-zingiberene, ar-curcumene, arginine, ascorbic acid, asparagine, β-bisabolol, β-carotene, β-elemene, β-eudesmol, β-ionone, β-myrcene, β-phellandrene, β-pinene, β-selinene, β-sesquiphellandrene, β-sitosterol, (β-thujone, bornyl-acetate, boron, caffeic acid, calcium, camphene, camphor, capric acid, caprylic acid, capsaicin, caryophyllene, chavicol, chlorogenic acid, chromium, citral, citronellal, citronellal, cobalt, copper, cumene, curcumin, cystine, delphinidin, Δ-cadinene, elemol, ethyl acetate, ethyl-myristate, farnesal, farnesene, ferulic acid, furfural, γ-aminobutyric acid, γ-terpinene, geranial, geraniol, geranyl-acetate, gingerenone, glutamic acid, glycine, hexahydrocurcumin, histidine, isogingerenone-B, isoleucine, kaempferol, lecithin, limonene, linoleic acid, magnesium, manganese, methionine, mufa, myrecene, myricetin, myristic acid, neral, nerol, nerolidol, niacin, nickel, oleic acid, oxalic acid, p-coumaric acid, p-cymene, p-hydroxy-benzoic acid, palmitic acid, pantothenic acid, paradol, patchoulic alcohol, phenylalanine, quercetin, riboflavin, selenium, shikimic-acid, terpinen-4-ol, thiamin, tryptophan, vanillic acid, vanillin, zinc, and zingerone.

Also, mixtures of two or more of these active compounds may be employed.

Ginger can be incorporated in the described composition in many different forms including ginger extract, ginger powder, one or more active compounds of ginger and mixtures thereof. Also, for any specific active compound of ginger for which suitable synthesis routes are known, the active compound can be prepared synthetically. Preferably, ginger is included in the composition as an extract of ginger. The ginger used in the described compositions may be used for flavoring purposes.

Any appropriate method can be used to prepare ginger extract. For example, ginger extract can be made by hydrodistillation, steam distillation, leaching, solvent extraction, pressing and extraction with supercritical carbon dioxide.

In a preferred embodiment, a powder prepared from ginger root extract may be processed by an unique condition to enhance shogaols. In certain preferred embodiments, the ginger extract contains 5% gingerols and 1.5% shogaols.

### Turmeric

Turmeric is a flowering plant, Curcuma longa, of the ginger family, Zingiberaceae.

Turmeric powder is about 60-70% carbohydrates, 6-13% water, 6-8% protein, 5-10% fat, 3-7% dietary minerals, 3-7% essential oils, 2-7% dietary fiber, and 1-6% curcuminoids. The golden yellow color of turmeric is due to curcumin.

Phytochemical components of turmeric include diarylheptanoids, a class including numerous curcuminoids, such as curcumin, demethoxycurcumin, and bisdemethoxycurcumin. Some 34 essential oils are present in turmeric, among which turmerone, germacrone, atlantone, and zingiberene are major constituents.

Curcumin is often sold as an herbal supplement, cosmetics ingredient and as food flavoring and food coloring, thus being safe for human consumption.

Mixtures of two or more of active compounds may be employed.

Turmeric can be incorporated in the described composition in many different forms including turmeric extract, turmeric powder, one or more active compounds of turmeric and mixtures thereof. Also, for any specific active compound of turmeric for which suitable synthesis routes are known, the active compound can be prepared synthetically. Preferably, turmeric is included in the composition as an extract of turmeric.

Any appropriate method can be used to prepare turmeric extract and will be known to those skilled in the art.

### Gingko Biloba

Ginkgo biloba, commonly known as ginkgo or gingko, also known as the maidenhair tree, is a species of tree native to China. It is the last living species in the order Ginkgoales.

Extracts of ginkgo leaves contain phenolic acids, proanthocyanidins, flavonoid glycosides, such as myricetin, kaempferol, isorhamnetin, and quercetin, and the terpene trilactones ginkgolides and bilobalides. The leaves also contain unique ginkgo biflavones, alkylphenols, and polyprenols.

Ginkgo biloba can be incorporated in the described composition in many different forms including Ginkgo biloba extract, Ginkgo biloba powder, one or more active compounds of Ginkgo biloba and mixtures thereof. Also, for any specific active compound of Ginkgo biloba for which suitable synthesis routes are known, the active compound can be prepared synthetically. Preferably, Ginkgo biloba is included in the composition as an extract of Ginkgo biloba.

Any appropriate method can be used to prepare Ginkgo biloba extract and will be known to those skilled in the art.

### Fennel

Fennel (Foeniculum vulgare) is a flowering plant species in the carrot family. It is a hardy, perennial herb with yellow flowers and feathery leaves. It is indigenous to the shores of the Mediterranean but has become widely naturalized in many parts of the world, especially on dry soils near the sea-coast and on riverbanks.

Fennel contains essential oil (e.g., anethole). It also contains not more than 10% estragole and not more than 7.5% fenchone. Other minor constituents may be present including: R-pinene, limonene, β-pinene, β-myrcene, and p-cymene. Furthermore, sweet fennel contains other nonvolatile constituents such as flavonoids and coumarins. The major components of fennel are phenylpropanoid derivatives: trans-anethole and estragole (methyl chavicol), and then alpha-phellandrene, limonene, fenchone, and alpha-pinene.

Fennel can be incorporated in the described composition in many different forms including fennel extract, fennel powder, one or more active compounds of fennel and mixtures thereof. Also, for any specific active compound of fennel for which suitable synthesis routes are known, the active compound can be prepared synthetically. Preferably, fennel is included in the composition as an extract of fennel.

Any appropriate method can be used to prepare fennel extract and will be known to those skilled in the art.

### Dong Guai

Angelica sinensis, commonly known as “dong quai” or “female ginseng,” is an herb belonging to the family Apiaceae, indigenous to China. Angelica sinensis grows in cool high-altitude mountains in East Asia. The yellowish-brown root of the plant is harvested in the fall and is used to prepare the extract of dong guai.

Over 70 compounds have been identified from dong quai, including essential oils, such as ligustilide, butylphthalide and senkyunolide A, phthalide dimers, organic acids and their esters such as ferulic acid, coniferyl ferulate, polyacetylenes, vitamins and amino acids. Z-ligustilide (water insoluble and heat stable), among which Z-butylidenephthalide and ferulic acid are thought to be the most biologically active components in Angelica sinensis.

The main chemical constituents of Angelica roots include ferulic acid, Z-ligustilide, butylidenephthalide and various polysaccharides.

Also, dong quai contains many organic acids. For example, ferulic acid isolated from dong quai is widely used as the marker compound for assessing the quality of dong quai and its products. Methanol, methanol-formic acid (95:5), 70% methanol, 70% ethanol, 50% ethanol or diethyl ether-methanol (20:1) may be used as the initial extraction solvent. The amount of ferulic acid in dong quai varies between 0.21 and 1.75 mg/g dry weight.

Dong quai can be incorporated in the described composition in many different forms including dong quai extract, dong quai powder, one or more active compounds of dong quai and mixtures thereof. Also, for any specific active compound of dong quai for which suitable synthesis routes are known, the active compound can be prepared synthetically. Preferably, dong quai is included in the composition as an extract of dong quai.

Any appropriate method can be used to prepare dong quai extract and will be known to those skilled in the art.

### Pine Bark

Another ingredient of the described composition may be an ingredient or an extract from the French maritime pine. French Pine extract is obtainable from the Pinus pinaster species of plants that belong to the Pinaceae family. Any appropriate method can be used to prepare French Pine extract. For example, French Pine extract can be made using any suitable extraction process known in the art, for example the process as described in U.S. Pat. No. 4,698,360. Other appropriate method can be used to prepare pine bark extract as well and will be known to those skilled in the art. For example, maritime pine bark extract can be made by first grinding pine bark, then washing and soaking it in hot water. Next, the solids are removed from the liquid extract. The liquid extract can then be used as is or further processed by freeze-drying and grinding it into a powder.

Such extracts can be standardized based on the extraction ratio, for example, 1000:1 i.e. 1000 parts pine bark to extraction media. Preferably, this ratio of pine bark to extraction media is used. French Pine extract can be obtained commercially from various herbal extract suppliers.

Maritime pines are known to contain health-promoting plant compounds like vitamins, polyphenols, and other phytonutrients. The most notable polyphenol nutrients in pine bark extract include procyanidins, catechins, and phenolic acids.

Pine bark can be incorporated in the described composition in many different forms including pine bark extract, pine bark powder, one or more active compounds of pine bark and mixtures thereof. Also, for any specific active compound of pine bark for which suitable synthesis routes are known, the active compound can be prepared synthetically. Preferably, pine bark is included in the composition as an extract of pine bark.

Any suitable process may be used for the preparation of a pharmacologically, nutraceutically or biologically active plant extracts substantially in a convenient administrable dosage form from the plants mentioned above.

Solvents useful for extracting plant material include water, ethanol, propanol, paraffin, hexane, petroleum ether, toluene, acetone, methyl ethyl ketone, and other common organic solvents. Water, ethanol, acetone and petroleum ether are the preferred solvents for use in plant extraction.

Alternatively, extracts and/or one or more of the active compounds contained therein can be purchased from commercial sources.

### III. Additional Non-Active Components of the Composition

In certain embodiments, the described composition may comprise a “vehicle” or “carrier,” which is preferably an inert substance. The function of the vehicle is to facilitate the incorporation of other compounds, to allow a better dosing and administration or to give consistency and shape to the composition. Therefore, the vehicle is a substance that is used to dilute any of the components of the described composition to a determined volume or weight, or even without diluting said components it is capable of allowing better dosing and administration or giving consistency and shape to the composition (e.g., nutraceutical composition). Due to all of this, a vehicle would be considered pharmaceutically or nutraceutically acceptable.

When the described composition is presented in liquid form, the vehicle is the diluent.

The compositions can also include one more excipients that are non-toxic and non-inflammatory in a subject.

In some embodiments, the excipient(s) can provide desirable or improved physical and/or chemical properties such as stability, flow, viscosity, rate of disintegration, taste, delivery, etc.

The composition described herein may additionally include, for example, electrolytes (e.g., potassium salt or other salts), sweeteners, flavoring and coloring agents, vitamins, minerals, preservatives, and antioxidants.

### Viscosity and Viscosity Modifiers

Viscosity is the ratio of shear stress to shear rate, expressed as dynes-second/cm², or poise. A centipoise (cP) is one one-hundredth of a poise.

The composition of the present invention may have a viscosity greater than water (i.e., about 1.0 cP at 20° C.), e.g., about 1.00, 200, 300, 400, 500, 1000, 1500, 2000, 2500, 3000, 3500, 4000, 4500, 5000, 6000, 7000, 8000, 9000 cP or more. if a consistency of corn syrup is desired, viscosities in the range of about 2500 cP are suitable. If a consistency of a soft gel or honey is desired, viscosities in the range of about 10000 cP to about 15000 cP are suitable. For pudding-like products, viscosities in the range of about 30000 cP to about 38000 cP are desirable. Viscosity of the compositions of the present invention may be measured with, e.g., a rheometer or viscometer, though additional methods of measuring viscosity are known in the art.

Viscosity modifiers may be added to the described compositions. Such viscosity modifiers include, for example, collagen, gellan gum, carbohydrate gel-forming polymers, carob bean gum, locust bean gum, carrageenan, alginates (e.g., alginic acid, sodium alginate, potassium alginate, ammonium alginate, and calcium alginate), agar, guar gum, xanthan aura, carboxymethyl cellulose, clear starch, pectin, gelatin, arrowroot, cornstarch, katakuri starch, potato starch, sago, tapioca, furcellaran, and sodium pyrophosphate. A viscosity modifier may be present in the composition in an amount of from about 0.01% to 10% by weight based on the total volume of the composition (e.g., 0.01, 0.1, 0.5, 1, 2, 3, 4, 5, 6, 7, 8, 9, or 10%), though the viscosity modifier may be present in lower or higher concentrations.

### Electrolytes

Exemplary electrolytes include potassium salts, chloride salts, bromide salts, sodium salts, magnesium salts, calcium salts, citrate salts, acetate salts, phosphate salts, salicylates, bicarbonate salts, lactate salts, sulphate salts, tartrate salts, benzoate salts, selenite salts, molybdate salts, iodide salts, oxides, and combinations thereof. An electrolyte may be present in a composition of the invention at a concentration range of about 0.01% to 10% by weight based on the total volume of the composition (e.g., 0.01, 0.02, 0.03, 0.04, 0.05, 0.1, 0.5, 1, 2, 3, 4, 5, 6, 7, 8, 9, or 10%), though an electrolyte may be present in lower or higher concentrations.

In certain embodiments, the compositions of the present invention include high concentrations of potassium (e.g., potassium chloride). The concentration of potassium in the composition may be, e.g., 0.01, 0.02, 0.03, 0.04, 0.05, 0.1, 0.5, 13, 4, 5, 6, or 7% or more by weight based on the total volume of the composition.

In certain embodiments, the compositions of the present invention include high concentrations of magnesium (e.g., magnesium chloride). The concentration of magnesium in the composition may be, e.g., 0.01, 0.02, 0.03, 0.04, 0.05, 0.1, 0.5, 1, 2, 3, 4, 5, 6, or 7% or more by weight based on the total volume of the composition.

### Sweeteners

Sweeteners may be included in the described compositions. Exemplary sweeteners include high fructose corn syrup, mannose, maltose, glucose polymers, sucrose (e.g., cane sugar or beet sugar), glucose, dextrose, lactose, galactose, fructose, polysaccharides (e.g., malodextrins), rice syrup, honey, and natural fruit juices (e.g., orange juice, papaya juice, pineapple juice, apple juice, grape juice, apricot juice, pear juice, tomato juice, agave nectar, or cranberry juice). Additionally, non- or low-caloric sweeteners can be used in the compositions of the invention. Examples of such non-caloric or low-caloric sweeteners include, but are not limited to, saccharin, cyclamates, acetosulfam, sorbitol, sucralose, xylitol, erythritol, Stevia, Stevia extract, L-aspartyl-L-phenyl-alanine ester (e.g., aspartame), L-aspartyl-D-alanine alkyl amides, L-aspartyl-L-1-hydroxymethylalkaneamide, and L-aspartyl-1-hydroxyethylalkaneamide.

Sweeteners may be present in a composition of the invention at a concentration range of about 2% to 20% by weight based on the total volume of the composition (e.g., 2, 3, 4, 5, 6, 7, 8, 9, 10, II, 12, 13, 14, 15, 16, 17, 18, 19, or 20%), though sweeteners may be present in lower or higher concentrations.

### Flavoring and Coloring Agents

Exemplary flavoring agents include natural and synthetic flavoring agents, including almond oil, amaretto oil, anethole, anise oil, benzaldehyde, blackberry, black walnut oil, blueberry, caraway, caraway oil, cardamom oil, cardamom seed, cherry juice, cherry syrup, cinnamon, cinnamon oil, cinnamon water, citric acid, citric acid syrup, clove oil, cocoa, coriander oil, dextrose, eriodictyon, ethyl acetate, ethyl vanillin, fennel oil, ginger, glucose, glycerin, glycyrrhiza, grape, honey, lavender oil, lemon oil, lime, mannitol, methyl salicylate, myristica oil, orange oil, orange peel, orange syrup, peppermint, peppermint oil, peppermint water, phenylethyl alcohol, pineapple, raspberry juice, raspberry syrup, rosemary oil, rose oil, rose water, sarsaparilla syrup, sorbitol, spearmint, spearmint oil, strawberry, sucrose, thyme oil, tolu balsam, tropical, vanilla, vanillin, and wild cherry syrup. Additional flavoring agents may be found in Food Chemicals Codex and Fenaroli's Handbook of Flavor Ingredients.

Small amounts of one or more coloring agents may be utilized in the described compositions. Coloring agents include, e.g., beta-carotene, riboflavin dyes, FD&C dyes (e.g., Yellow No. 5, Blue No. 1, Blue No. 2, and Red No. 40), FD&C lakes, chlorophylls and chlorophyllins, caramel coloring, annatto, cochineal, turmeric, paprika, and fruit, vegetable, and/or plant extracts (e.g., grape, black currant, aronia, carrot, beetroot, red cabbage, elderberry, and hibiscus extracts). The amount of coloring agent used will vary depending on the agents used in the composition and the color intensity desired in the finished product. The amount of coloring agent to be used can be readily determined by one skilled in the art.

In some embodiments, saffron in the described composition may function as a sweetener.

### Vitamins and Minerals

Non-limiting examples of vitamins and minerals that may be included in the described compositions include, e.g., choline bitartate, niacinamide, thiamin, folic acid, d-calcium pantothenate, biotin, vitamin A, vitamin C, vitamin B₁ hydrochloride, vitamin B₂, vitamin B₃, vitamin B₆ hydrochloride, vitamin B₁₂, vitamin D, vitamin E acetate, vitamin K, and salts of calcium, potassium, magnesium, zinc, iodine, iron, and copper. When included in the described composition, the composition contains at least 5, 10, 15, 20, 25, 30, 35, 40, 45, or 50% of the U.S. recommended daily intake (RDI) for such vitamins and minerals.

### Preservatives

One or more preservatives may additionally be utilized in the described compositions. Exemplary preservatives include, for example, sorbate, benzoate, and polyphosphate preservatives (e.g., sorbic acid, benzoic acid, calcium sorbate, sodium sorbate, potassium sorbate, calcium benzoate, sodium benzoate, potassium benzoate, and mixtures thereof).

In certain embodiments, the described composition may also include additional maltodextrin (i.e., in addition to the maltodextrin used to encapsulate the ingredient or the extract of saffron). Maltodextrin is generally used as a thickener or filler to increase the volume of a processed food. It's also a preservative that increases the shelf life of packaged foods.

When included in a composition of the invention, the preservative is included at levels from about 0.0005% to about 0.5% (e.g., 0.0005, 0.001, 0.005, 0.01, 0.05, 0.1, or 0.5%) by weight based on the total volume of the composition, though preservatives may be present in lower or higher concentrations.

### Antioxidants

One or more antioxidant agents may also be included in the described compositions. Exemplary antioxidants include vitamin C and vitamin E; beta-carotene, lutein, or other carotenoids; cyanidin, delphinidin, malvidin, or other anthocyanidins; apigenin, luteolin, or other flavones; hesperitin, naringenin, or other flavonones; isorhamnetin, quercetin, kaempferol or other flavonols; and epigallocatechin-3-gallate, epicatechin, thearubigins, or other flavan-3-ols.

Additional components of the compositions described herein may include amino acids (e.g., leucine, isoleucine, lysine, methionine, phenylalanine, threonine, tryptophan, and valine), stimulants (e.g., caffeine), emulsifying agents, carbon dioxide (e.g., to carbonate a liquid composition), stabilizers, humectants, anticaking agents, or herbal extracts not previously mentioned.

In certain embodiments, the described compositions may include Medium Chain Triglyceride (MCT) oil. For example, coconut oil is a type of MCT oil. It is of a certain chain length that is different from saturated fats (longer) or free fatty acids (shortest).

Importantly, the additional components are pharmaceutically and or nutraceutically acceptable.

In certain preferred embodiments, the described composition includes safe, pure, and effective ingredients as determine by Nutrilite™ Quality Standards and Traceability program.

In certain embodiments, the described composition does not contain any GMO ingredient(s).

In certain embodiments, the composition may be lactose free.

In certain embodiments, the composition may be diary free.

In certain embodiments, the composition may be soy free.

In certain embodiments, the composition may be gluten free.

In certain embodiments, the composition may not contain artificial sweeteners.

In certain embodiments, the composition may not contain artificial preservatives.

In certain embodiments, the composition may not contain artificial flavors.

In certain embodiments, the described composition may not contain artificial colors.

Also, in certain embodiments, no refrigeration is necessary for the described composition (e.g., can be stored in a cool, dry place).

Furthermore, the described composition may not include irradiated ingredients.

In certain embodiments, the described composition may be Kosher and/or Hala certified.

In certain embodiments, the described composition may be vegetarian and/or vegan.

In certain embodiments, the described composition may be NSF certified.

In certain embodiments, the described composition may be free from peanuts, tree nuts, eggs, shellfish, and crustacean.

### IV. Combination Therapies

In certain embodiments, the described composition may comprise other active substances. In addition to the requirement of therapeutic effectiveness, where said composition may require the use of other therapeutic agents, there may be additional fundamental reasons that compel or strongly recommend the use of a combination of a described composition and another therapeutic agent. The term “active substance” or “active agent” is any matter, regardless of its origin, human, animal, plant, chemical or other, to which a specific activity is attributed in order to constitute a medicament.

In each case, the form of presentation of the medicament will be adapted to the chosen route of administration. Thus, in certain embodiments, the described composition may be presented in the form of solutions or any other form of clinically permitted administration and in a therapeutically effective dosage. For example, the composition may be presented in solid, semi-solid, liquid or gaseous form; as a tablet, capsule, powder, granule, ointment, solution, suppository, injection, inhalant, gel syrup, nebulizer, microsphere or aerosol. Preferably, the composition is adapted for oral administration, for example in the form of a tablet, capsule, powder, granule solution or syrup.

In certain embodiments, additional therapeutic agent(s) may be administered with the described compositions for, e.g., the treatment or amelioration of menstrual symptoms or discomforts. Such therapeutic agents include, for example, muscle relaxants (e.g., diazepam) or anti-inflammatory agents (e.g., ibuprofen). When combination therapy is employed, the additional therapeutic agent(s) can be administered as a separate formulation or may be combined with any of the compositions described herein.

For example, in some embodiments, the compositions can be used in combination with one or more sleep aids. Sleep aids that can be used in combination with the compositions and methods described herein include: antihistamines (e.g., diphenhydramine and doxylamine); benzodiazepines estazolam (ProSom), flurazepam (Dalmane), quazepam (Doral), temazepam (Restoril), and triazolam (Halcion)); non-benzodiazepine sedative hypnotics (e.g., eszopiclone (Lunesta), zalepon (Sonata), and zolpidem (Ambien)); and melatonin receptor agonist hypnotics (e.g., ramelteon (Rozerem). Still other sleep aids that can be used in combination with the compositions and methods described herein include: chamomile, valerian root, kava, lemon balm, passionflower, lavender, St. John's Wort, melatonin, tryptophan (e.g., L-tryptophan), 5-hydroxytryptophan (5-HTP), catnip, hops, rhodiola, oatstraw, lavender, GABA, L-theanine, linden, ginseng (e.g., Siberian ginseng), honey, nutmeg, mugwort, butterbur, rauwolfia, taumelloolch, American hellebore, quassia, tulip tree, brewer's yeast, inositol, skullcap, phosphatidylserine, calcium, magnesium, vitamin B6, vitamin B12, and pantothenic acid (B5).

### V. Formulations and Methods of Preparing Compositions

The compositions and solutions described herein may be formulated as ready-to-drink beverages, concentrates (e.g., syrups), dry compositions (e.g., powders, granules, or tablets that may be reconstituted with a liquid (e.g., with water), gels, solids, semi-solids (e.g., ice cream, pudding, or yogurt), frozen liquids (e.g., ice pops), lozenges or hard candies, dissolving strips (e.g., an edible strip containing pullulan and compositions of the invention), and chewing gum.

In certain embodiments, compositions and solutions described herein may be formulated as a food supplement. The term “food supplement” is understood as the products marketed in the form of capsules, tablets, phials, herbal infusions, drinkable solutions, etc., intended to supplement the usual diet and which constitute a concentrated source of nutrients (such as vitamins, minerals, amino acids, essential fatty acids, fiber, etc.) or other substances that have a nutritional, or physiological effect.

In some embodiments, the compositions may be in the form of a dry powder, granule, or tablet that may be reconstituted in a specified amount of a liquid. The dried components may be mixed together and milled (e.g., to create a homogenous powder) or mixed in aqueous solution and dried by using methods known to one of skill in the art. Dried powders or granules may be “loose” or fashioned into tablets.

The compositions and solutions described herein can be ingested (e.g., through eating or drinking) throughout the menstrual cycle, before the onset of menstrual symptoms or discomforts, when menstrual symptoms or discomforts begins, any time after the onset of menstrual symptoms or discomforts, or after menstrual symptoms or discomforts have subsided. When the compositions and solutions of the present invention are in the form of a ready-to-drink beverage, e.g., 1, 2, 4, 8, 10, 12, 14, 16, 18, 20, 24, 26, 28, 30, or 32, or more ounces of the beverage may be consumed as needed (e.g., once, twice, three, four, five, six times per day; once, twice or multiple times per week; or once, twice, multiple times, or daily per month).

For example, in certain embodiments, to prevent, alleviate, ameliorate, or treat common menstrual cycle symptoms or discomforts, the described composition may be consumed daily for a minimum of 1 menstrual cycle; alternatively, for a minimum of 2 menstrual cycles; alternatively, for a minimum of 3 menstrual cycles; and alternatively, for a minimum of 6 menstrual cycles.

The compositions and solutions of the present invention may be prepared using methods known to one of skill in the art. Such methods include dissolving, dispersing, or otherwise mixing all components singularly or in suitable combinations and agitating with, for example, a mechanical stirrer until all of the ingredients have been solubilized or adequately dispersed. Where a shelf-stable composition or solution is desired, the final mixture can be pasteurized, ultra-pasteurized, sterilized, or filled aseptically at appropriate process conditions. Where required for mutual stability of two or more components (for example if a component is unstable at low pH), multiple components can be mixed shortly before ingestion.

In one embodiment, the described composition is a powdered drink mix or capsule or tablet intended for daily consumption for a minimum of 2 menstrual cycles (i.e., approximately 2 months) but it is intended for long-term, daily use. The powdered drink mix can be consumed with cold or warm water or mixed with other beverages such as smoothies and carbonated drinks.

The compositions and solutions described herein may be bottled or packaged in, for example, glass bottles, plastic bottles and containers (e.g., polyethylene terephthalate or foil-lined ethylene vinyl alcohol), metal cans (e.g., coated aluminum or steel), lined cardboard containers, pouches, packs, wrappers, or any other packaging known to one of skill in the art. For example, a ready-to-drink beverage can be bottled or packaged in a unit that contains between 10-1000 mL of the beverage. For example, the packaging can contain 10, 20, 30, 40, 50, 100, 200, 300, 400, 500, 600, 700, 800, 900, or 1000 mL of the beverage. Alternatively, the packaging can contain 200, 250, 330, 350, 355, 375, 440, or 500 mL of the beverage. A ready-to-drink beverage can also be bottled or packaged in a unit that contains between 1-32 fluid ounces of beverage (e.g., the unit may contain 1, 2, 5, 6.75, 8, 8.3, 8.4, 8.45, 9.6, 10, 12, 15, 15.5, 16, 18.6, 20, 23, 24, or 32 fluid ounces). Where a shelf-stable composition or solution is desired, the packaging is appropriately sterilized before being filled by the pasteurized, ultra-pasteurized, or sterilized composition or solution. Where required for mutual stability of two or more components (for example if a component is unstable at low pH), the packaging may feature multiple containers that can be mixed shortly before ingestion or that can be consumed serially.

In certain embodiments, one menstrual health beverage may be one serving, which may include 10-500 mg of the ingredients per beverage; alternatively, one menstrual health beverage may be one serving, which may include 10-400 mg of the ingredients per beverage; alternatively, one menstrual health beverage may be one serving, which may include 10-350 mg of the ingredients per beverage; alternatively, one menstrual health beverage may be one serving, which may include 10-300 mg of the ingredients per beverage; alternatively, one menstrual health beverage may be one serving, which may include 50 mg of the ingredients per beverage; alternatively, one menstrual health beverage may be one serving, which may include 75 mg of the ingredients per beverage; alternatively, one menstrual health beverage may be one serving, which may include 100 mg of the ingredients per beverage; alternatively, one menstrual health beverage may be one serving, which may include 200 mg of the ingredients per beverage; alternatively, one menstrual health beverage may be one serving, which may include 300 mg of the ingredients per beverage; alternatively, one menstrual health beverage may be one serving, which may include 400 mg of the ingredients per beverage; alternatively, one menstrual health beverage may be one serving, which may include 500 mg of the ingredients per beverage. Amounts in-between the listed amounts are also contemplated and can be used to produce the described health beverage.

In certain embodiments, instructions can be included with the described composition. The instructions, e.g., may be as follows: For adults: Add contents of one stick pack to 150-250 ml of water and stir. For a stronger flavor, less water can be used and for a milder flavor more water can be used. May be mixed with warm or cold beverages such as water or juice. For best results, consume one serving daily.”

### VI. Uses

The described compositions and methods may be used to ease and/or alleviate common menstrual cycle symptoms and discomforts.

As discussed previously, common menstrual symptoms and discomforts include physical, physiological, and/or physiological symptoms and discomforts, including any combinations of these symptoms and/or discomforts.

Physical symptoms associated with the menstrual cycle—but not necessarily with PMS, which is symptoms that appear before menstruation, rather than during menstruation—include bloating, abdominal discomfort, lower back pain, cramps in the pelvic area, constipation/diarrhea, swelling or tenderness in the breasts, cyclic acne (acne that appears and disappears at predictable times during the menstrual cycle), joint or muscle pain, and food cravings. The exact symptoms and their intensity vary significantly from person to person, and even somewhat from cycle to cycle and over time. Most people with premenstrual syndrome experience only a few of the possible symptoms, in a relatively predictable pattern.

Examples of physiological symptoms may include but are not limited to, e.g., the physical symptoms that occur when one feels anxious or under display, e.g., higher than normal heart rate and blood pressure. These are bodily reactions, and may be apparent to other people. Note that they are always stronger and more apparent to the person exhibiting them than to the person displaying them.

Examples of psychological or emotional symptoms may include but are not limited to, e.g., stress, anxiety, depression, difficulty with sleep, headache, feeling tired, mood fluctuations/swings, increased emotional sensitivity, and changes in interest in sex, or any additional symptoms associated with normal menstrual health cycles. Problems with concentration and memory may also be other examples of psychological or emotional symptoms.

### VII. Methods

Within the context of the present invention, methods of treatment equivalent to the uses described in the present description are also contemplated. Thus, in another aspect, the invention relates to a method for preventing symptoms and/or discomforts related to menstrual cycle in a subject that comprises administering the described composition to said subject as described above.

In another aspect, the invention relates to a method for alleviating symptoms and/or discomforts related to menstrual cycle in a subject that comprises administering the described composition to said subject as described above.

In another embodiment, described herein is a method of preventing and/or ameliorating menstrual cycle symptoms and/or discomforts in a subject in need thereof, comprising orally consuming an effective amount of the described composition to prevent and/or ameliorate the subject's menstrual cycle symptoms or discomforts.

In yet further embodiment, described herein is a method of preventing and/or ameliorating menstrual cycle symptoms and/or discomforts in a subject in need thereof, comprising orally consuming an effective amount of (a) an extract of chasteberry fruit; (b) an extract of saffron red sargol stigma, wherein the extract of saffron red sargol stigma is encapsulated by maltodextrin, to prevent and/or ameliorate the subject's menstrual cycle symptoms or discomforts. The ingredients (a) and (b) provide prevention and/or amelioration of the menstrual cycle symptoms and/or discomforts, wherein the menstrual cycle symptoms and/or discomforts comprise physical, physiological and/or psychological symptoms or discomforts experienced during the monthly menstrual cycle of the patient. The physical menstrual symptoms and/or discomforts may be bloating, abdominal discomfort, lower back pain, cramps in the pelvic area, constipation, diarrhea, swelling or tenderness in the breasts, cyclic acne, joint or muscle pain, and food cravings. The psychological symptoms and/or discomforts may be stress, anxiety, difficulty with sleep, headache, feeling tired/fatigue, mood swings, increased emotional sensitivity, changes in interest in sex, problems with concentration and memory, and depression.

In the described method, the ingredients (a) and (b) may be orally consumed as a composition.

In the described method, the step of orally consuming the ingredients (a) and (b) may occur prior to, during, or after the onset of the menstrual cycle symptoms and/or discomforts.

In the described method, the step of orally consuming the ingredients (a) and (b) may occurs once or more times per day, and preferably daily.

In the described method, the step of orally consuming the ingredients (a) and (b) may occurs daily for a minimum of 1 menstrual cycle; more preferably, daily for a minimum of 2 menstrual cycles; more preferably, daily for a minimum of 3 menstrual cycles; more preferably, daily for a minimum of 6 menstrual cycles.

In the method, the subject may be orally consuming between about 20 to about 40 mg per day of the extract of chasteberry fruit, and between about 30 to about 60 mg per day of the extract of saffron red sargol stigma.

In the method, the composition may be consumed as a supplement.

In the method, the composition may be consumed as a food product.

### EXAMPLES

### Example 1: An Exemplary Product of the Present Invention

A product of the present invention was formulated in the form of capsule. The ingredients of the capsule are listed below.

Table 1 provides an exemplary composition of the described invention with amounts for each of the above extracts, and other components that are contained within 3 grams of the described composition, which would be 1 serving size.

TABLE I

| Ingredient | Formula Qty. (g) | Theor (%) |
|---|---|---|
| Fructose Food Grade IP | 1.4580 | 48.6000 |
| Citric Acid | 0.1100 | 3.6667 |
| Stevia | 0.0100 | 0.3333 |
| Nat Flavor, Tropical (e.g., | 0.0500 | 1.6667 |
| mango) | | |
| Nat Flavor, Tropical (e.g., | 0.0400 | 1.3333 |
| pineapple) | | |
| Saffron Extract | 0.0460 | 1.5333 |
| Ginger Extract | 0.0250 | 0.8333 |
| Chasteberry Extract | 0.0310 | 1.0333 |
| Maltodextrin | 1.2288 | 40.9600 |
| Medium Chain Triglyceride | 0.0012 | 0.0400 |
| (MCT) Oil | | |
| LOT TOTAL: | 3.0000 | 100.000 |

### Example 2: Consumer Testing and Prototypes (Prophetic Example)

Study conducted using chasteberry fruit and saffron stigma have yielded benefits to support menstrual health symptoms such as mood swings, abdominal cramps, and breast tenderness. Some of the benefits between the two ingredients may overlap however there is no research to support the hypothesis. Amway Business Group (ABG) is sponsoring a clinical study to evaluate the safety and the tolerance of the menstrual health drink powder (formula).

The clinical study is a two-arm, double-blind, randomized clinical trial that is recruiting up to 125 women with at least 50 women completing each arm (placebo and test). Each participant will consume either the menstrual health formula (test) or the placebo formula with matching flavoring and color daily for 3 months with a washout for 1 month for a total of 4 months. The menstrual health formula is composed of 30 mg chasteberry fruit extract, 45 mg saffron stigma extract, and ginger for flavoring intended to be mixed with water for consumption. Participants being recruited are between 18 to 50 years old, who are healthy and experience monthly menstruation with consistent physical and emotional symptoms.

In addition to blood tests, participants are offering daily and monthly feedback in response to the consumption of the assigned product. Feedback include a daily diary, Premenstrual Syndrome Questionnaire, Tolerability Questionnaire, and Consumption Behavior Questionnaire. The feedback aims to collect how the participants feel about consume the product (placebo or test), any changes in menstruation cycle and symptoms, and if the symptoms return during washout period. We hypothesize that the menstrual health formula is safe to consume daily, long-term. In addition, we predict that daily consumption of saffron and chasteberry extracts can, a minimum, ease mood swings, abdominal cramps, and breast tenderness. We anticipate that a select number of participants will also experience additional benefits not listed above.

### Example 3

### Background

Inflammation is a defense mechanism involving a cascade of reactions. Casimir et al. found that there is a biological sex influence on inflammatory response in males and females, i.e., differences in how males and females respond to inflammatory and painful stimuli (Casimir, G. J., et al., 2013. 4: p. 1-9). For example, males can experience an increased pathogen load and reduced immune function when compared to females. The prevalence of acute inflammatory disease is greater in men as compared to women. Past studies showed that females often experience better prognosis and lower mortality rates due to acute infectious diseases (Haider, A. H., et al., Surgery, 2009. 146(2): p. 308-315). The genetic make-up of females may provide insights why this may be true.

Female cells have two X chromosomes where females possess genes from paternal X chromosome and maternal X chromosome. Because of the multiple copies of the X chromosomes in female cells, females possess polymorphism of X-linked gene providing females greater diversity to fight against infection challenges compared to the uniform cell population in males (Haider, A. H., et al., Surgery, 2009. 146(2): p. 308-315).

X-inactive specific transcript (XIST) is a 17-kb spliced, polyadenylated long non-coding RNA (IncRNA) produced from one of two X chromosomes in female cells. XIST is retained within the nucleus and coats the X chromosome of origin leading to chromosomal inactivation (Shenoda, B. B., et al., Cellular and Molecular Life Sciences, 2021. 78: p. 299-316). In addition to silencing one of the X chromosome of the pair, XIST has been linked to inflammatory process explaining the potential difference in reaction between men and women.

A study conducted by Shenoda et al. demonstrated that the possession of XIST gene can have a protective anti-inflammatory effect on female cells. The study was conducted using in-vivo and in-vitro inflammatory models in mouse and human cells, respectively. The results showed that XIST can increase gene expression in the cytoplasm of the cell and therefore delay nuclear factor-kB (NF-kB) and slow inflammatory response. NF-kB is a transcription factor that regulates multiple aspects of inflammation and immunity.

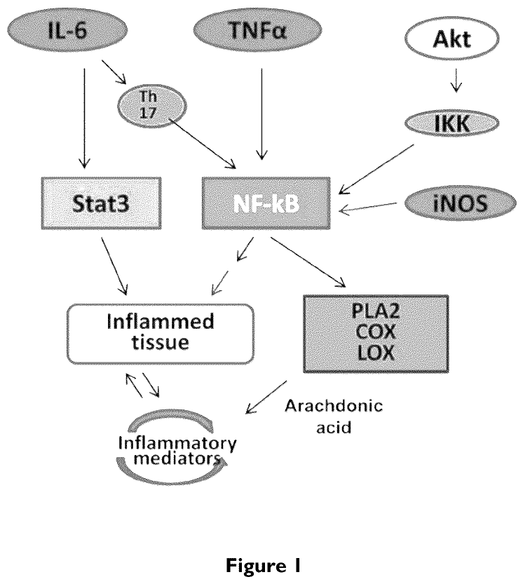

The arachidonic acid pathway is a downstream signaling of NF-kB where arachidonic acid is a lipid mediator that is oxidized by cyclooxygenase (COX) and lipoxygenase (LOX) to produce proinflammatory mediators called eicosanoids (Chen, S., Current Drug Targets, 2011. 12(3): p. 288-301) (FIG. 1). Two eicosanoids of interest to female health are prostaglandin and leukotrienes that play a vital role in inflammatory states during premenstrual syndrome (Li, C., et al., Molecular Medicine Reports, 2019. 19(1): 727-733). Physically, these chemical changes manifest in uterine pain and abdominal contractions, which are chief complaints during women's monthly menstrual cycles.

This study was designed to look at the XIST gene, as its regulation may have an impact on women that differentiate their experience from men. It was hypnotized that an upregulation of this gene may offer some temporary relief for women during their menstrual cycle.

### Methods

The study was designed to look at the global gene expression and to assess any gene expression differences in cells treated with Chasteberry or Saffron alone, or a blend of the two ingredients in the transcriptomics experiment. The experiment is outlined below.

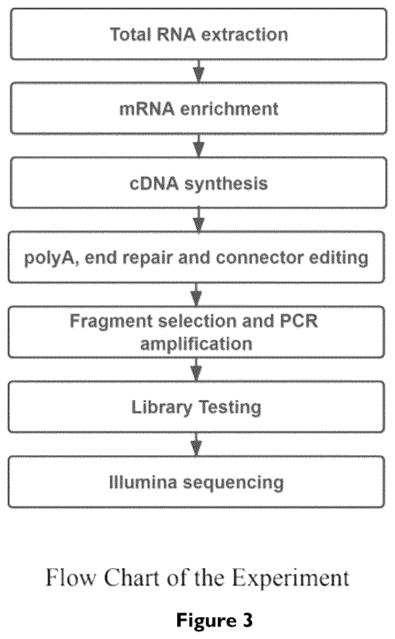

Transcriptome broadly refers to the collection of all transcripts in a cell under a certain physiological condition. The workflow of transcriptome sequencing experiment includes RNA extraction, RNA detection, library construction, and sequencing. The flow-chart is shown in FIG. 3.

In this experiment HepG2 cell line was used and the cells were subjected to four different treatments as follows:
Control (untreated cell)
Chasteberry only
Saffron only
Chasteberry and Saffron in 6:9 ratio (i.e., 30 mg to 45 mg).

The time for treatment was 18 hrs. After treatment cells were processed and total RNA was extracted followed by the steps as shown in FIG. 3. The test was carried out in only one ratio that was identical to that of the described formula.

### Results

Cells were treated with saffron only, chasteberry only, and a combination of chasteberry and saffron in a 6:9 ratio that equaled an exemplary drink formulation (30 mg chasteberry extract and 45 mg saffron extract).

Gene expression for XIST was seen for saffron only, chasteberry only, and combination of saffron and chasteberry.

Gene expression was quantified in folds (magnitudes) where saffron was compared to control (SA v CTRL), chasteberry was compared to control (CH v CTRL), saffron and chasteberry were compared to control (SA+CH v CTRL), saffron and chasteberry were compared to saffron (SA+CH v CTRL), and saffron and chasteberry were compared to chasteberry (SA+CH v CTRL). In addition to the gene expression quantified in folds, the inventors looked at whether the gene was qualitatively expressed as up for upregulations, down for downregulation, and false for false positives.

Results for XIST are summarized in Table 2 below.

SA v CTRL, XIST was falsely regulated by −3.768 folds, CH v CTRL XIST was falsely regulated by 6.288 folds and SA+CH v CTRL was upregulated by 7.32 folds. Although SA+CH v SA was upregulated by 11.099, because SA v CTRL and CH v CTRL were false, the quantification may not be meaningful. With SA v CTRL and CH v CTRL producing a false positive, we can assume a value of 1 or no difference from control therefore SA+CH v CTRL produced a 7.32 fold difference (Table 2).

TABLE 2

| Treatment | Log₂ Fold Change | p Value | Regulated |
|---|---|---|---|
| SA + CH + CTRL | 7.321 | 6.8501E−06 | UP |
| SA + CH + SA | 11.099 | 1.50335E−06 | UP |
| SA + CTRL | −3.768 | 0.151206 | FALSE |
| CH + CTRL | 6.288 | 0.00913 | FALSE |

### Conclusion

Women experience monthly menstrual cycle where fluctuation of hormones may be one of the contributing causes of premenstrual syndrome (PMS) symptoms. PMS symptoms are quite common with up to 90% of women experiencing at least one symptom during their lifetime. Abdominal cramps and uterine contractions are some of the more common symptoms. While the PMS mechanism needs additional exploration, scientists hypothesize that hormone changes trigger inflammatory pathways resulting in pain. Pain medication is the most common solution. However, currently described study surprisingly showed that a composition including the extracts of chasteberry and saffron may be used instead of pain medication to treat PMS in women. Specifically, the study showed that exposing cells to chasteberry and saffron together can increase the expression of XIST by more than 7-fold, when compared to control. XIST gene is hosted on the X chromosome and past studies in mouse and humans have shown an attenuation of the NF-kB. Conversely, mouse cells with XIST gene knocked out significantly influenced the expression of NF-kB pathway contributing to an acceleration of inflammatory cascade. The downstream effect of NF-kB is linked to inflammatory pathways and inflammatory mediators, such as arachidonic acid pathway and eicosanoids, respectively. Therefore, presently described study suggested that the consumption of chasteberry and saffron together in our suggested ratio may offer some physical relief during PMS.

### Example 4: A Pilot Study to Assess the Effects and Safety of a Supplement Versus Placebo in Alleviating Premenstrual Symptoms

Chasteberry extract used in this formula had an extraction ratio of 6:1 and was extracted with 50% ethanol. A clinical dose of 20-40 mg per day has been suggested to support common menstrual cycle symptoms when consumed daily for a minimum of 2 menstrual cycles. Most studies test an extraction ratio of 5:1, standardized to 3% crocin, and extracted with 30% ethanol. A clinical dose of saffron at 30 mg per day has been suggested to support normal mood changes during menstrual cycle symptoms when consumed for a minimum of 2 menstrual cycles. The proposed formula uses saffron stigma with an extraction ratio of 3.33:1, standardized to 2% crocin, and extracted with 30% ethanol; therefore, a dose of 45 mg per day was recommended to maintain the same level of crocin per daily dose. The saffron was processed with heat and a drying process that supports the preservation of key metabolites such as safranal and crocin.

This single-center, randomized, controlled pilot study was conducted to assess the efficacy, tolerability, and safety of the supplement, Women's Menstrual Health (drink powder), in easing PMS symptoms when used over the course of 3 months by healthy women with consistent menstrual cycles who regularly experience PMS symptoms, and to assess effects after a 1-month regression.

### Procedures and Methods

Prior to the start of the study, prospective subjects were prescreened for eligibility criteria using an IRB-approved script. Women 18 to 50 years of age having a regular, consistent menstrual cycle (between 25-35 days), who regularly experience premenstrual syndrome (PMS) symptoms, were scheduled for eligibility screening at the clinic±4 days of the start of the next expected menstrual cycle. Prospective subjects were advised to arrive at the clinic well-hydrated for blood draw procedure.

At visit 1 (baseline), prospective subjects completed the informed consent process and signed the ICF. Prospective subjects who signed this initial paperwork were assigned a screening number.

Prospective subjects were screened for eligibility criteria. Candidate subjects who pass the initial screening completed a urine pregnancy test (UPT). Each candidate subject's eligibility was reviewed, and qualified subjects (including a negative UPT result) were enrolled in the study and assigned a subject number.

Subjects participated in the following procedures:

### Blood Draw

Subjects had blood samples drawn to evaluate liver enzymes and CBC to ensure health and safety.

Blood draw procedure was performed at no expense to the subjects, either in the clinic or at an offsite location. Offsite blood draw was performed within 24 hours after subject's in-clinic procedures.

### Premenstrual Syndrome Questionnaire

Subjects completed a Sponsor-provided premenstrual syndrome questionnaire, rating the severity (on average) with which subject experienced the listed PMS symptoms in the week before and after her period, and severity of menstrual cramps and backache during the first two days of her period.

### Tolerability Questionnaire

Subjects completed a Sponsor-provided tolerability questionnaire, which included inquiries on subjects' past menstrual cycles and history of PMS product use.

In accordance with a predetermined randomization, subjects were assigned to cell 1 (Powder Supplement) or cell 2 (Placebo).

Subjects were provided with 70 units of the assigned test material. Subjects were also provided with oral and written usage instructions, a calendar of study visits, and a daily diary to record test material consumption time, use of OTC medication (NSAID, naproxen, acetaminophen, etc.), and comments.

Subjects were instructed to begin consuming the assigned test material immediately on the first day of their next period/menstruation. The first day of bleeding (including spotting) was deemed day 1 of the cycle.

Subjects were also instructed to complete two questions included in the daily diary (regarding what they liked and disliked about the test material) immediately after the first test material consumption; and to complete the premenstrual syndrome and tolerability questionnaires on a provided weblink at home at month 1 (±4 days).

Subjects returned to the clinic for visit 2 (month 2) and visit 3 (month 4). Subjects participated in the following procedures at each visit (unless otherwise indicated):

Clinic personnel recorded concomitant medications and questioned subjects regarding changes in their health. Aes were recorded if applicable.

Daily diaries were collected and reviewed for Aes and compliance (including completion of the two questions in the daily diary). A new daily diary was distributed to each subject at month 2 and completed diaries were retained by the clinic.

At month 2, any unused test material units were visually inspected for compliance and returned to subjects, and 22 new units were distributed to each subject. Subjects were instructed to stop consuming the test material at month 3; to complete two questions included in the daily diary (regarding what they liked and disliked about the test material) immediately after the last test material consumption; and to complete the premenstrual syndrome and tolerability questionnaires on a provided weblink at home at month 3 (±4 days). At month 4, any unused test material units were retained by the clinic.

Subjects completed the premenstrual syndrome and tolerability questionnaires at months 2 and 4, and the blood draw procedure at month 4, as previously described.

At month 4, subjects completed a Sponsor-provided consumption behavior questionnaire regarding daily consumption and likelihood of future use of the test product.

### Biostatistics and Data Management

Statistical analyses of the study data and data management were conducted in accordance with the protocol.

The statistical baseline for month 4 data was month 3 (regression baseline) (in addition to baseline).

### Quality Assurance

All clinical research studies performed in accordance with federal regulations and Good Clinical Practice guidelines. The independent Quality Assurance Unit monitored the study conduct and audited the study documents, data, and clinical study report. All data and supporting documentation are accurate, complete, and in compliance with the requirements of the protocol.

Data review and analyses were performed by an independent data committee, consisting of selected representatives from clinical services, quality assurance unit, and statistics department.

Of the 132 subjects enrolled in the study, 109 subjects were included in the intent-to-treat (ITT) population and 90 subjects completed study participation (per-protocol [PP] population).

For the ITT population, 55 subjects were in cell 1 (Powder Supplement) and 54 subjects were in cell 2 (Placebo). For the PP population, 46 subjects were in cell 1 and 44 subjects were in cell 2.

In accordance with a predetermined randomization, subjects were assigned to cell 1 or cell 2. During the usage period (first 3 months of the study: baseline through month 3), subjects consumed the assigned test material once per day as directed. During the regression period (last 1 month of the study: month 3 time point through month 4), subjects did not consume any of the assigned test material.

Evaluations were conducted in the clinic at visit 1 (baseline), visit 2 (month 2), and visit 3 (month 4), and at home at month 1 and month 3.

Table 3 presents the results of the tolerability questionnaire. Values at post-baseline time points were statistically compared to values at baseline for significant (P≤0.05) differences.

TABLE 3
Table 3: Results for Tolerability Questionnaire

| | Time | | | Subject | Subject | Mean | Mean | Signed Rank | |
|---|---|---|---|---|---|---|---|---|---|
| Treatment | Point | N | Mean | Increased, % | Decreased, % | Change | Change, % | Test | P Value^{a} |

What is your normal menstrual cycle length?
What is your menstrual cycle length this month?
1 = 21-25 days, 2 = 26-30 days, 3 = 31-35 days

| | | | | | | | | | |
|---|---|---|---|---|---|---|---|---|---|
| Women's | Baseline | 55 | 1.76 | | | | | | |
| Menstrual | Month 1 | 52 | 1.79 | 11.5 | 9.6 | 0.02 | 1.1 | 1.000 | — |
| Health | Month 2 | 48 | 1.79 | 16.7 | 14.6 | 0.02 | 1.2 | 1.000 | — |
| Powder | Month 3 | 39 | 1.95 | 25.6 | 7.7 | 0.21 | 11.8 | 0.076 | — |
| Supplement | Month 4 | 46 | 1.87 | 26.1 | 17.4 | 0.11 | 6.2 | 0.407 | — |
| Placebo | Baseline | 54 | 1.80 | | | | | | |
| | Month 1 | 51 | 1.65 | 11.8 | 23.5 | −0.12 | −6.7 | 0.307 | — |
| | Month 2 | 47 | 1.79 | 10.6 | 14.9 | −0.04 | −2.3 | 0.774 | — |
| | Month 3 | 41 | 1.68 | 19.5 | 29.3 | −0.12 | −6.8 | 0.407 | — |
| | Month 4 | 43 | 1.77 | 16.3 | 23.3 | −0.05 | −2.6 | 0.842 | — |

What is your normal period length?
What is your period length this month?
1 = 1-3 days, 2 = 4-6 days, 3 = ≥7 days

| | | | | | | | | | |
|---|---|---|---|---|---|---|---|---|---|
| Women's | Baseline | 55 | 2.00 | | | | | | |
| Menstrual | Month 1 | 52 | 2.08 | 15.4 | 5.8 | 0.10 | 4.9 | 0.227 | — |
| Health | Month 2 | 48 | 2.13 | 16.7 | 4.2 | 0.13 | 6.3 | 0.109 | — |
| Powder | Month 3 | 39 | 1.97 | 10.3 | 7.7 | 0.03 | 1.3 | 1.000 | — |
| Supplement | Month 4 | 46 | 2.02 | 13.0 | 10.9 | 0.04 | 2.2 | 0.795 | — |
| Placebo | Baseline | 54 | 2.04 | | | | | | |
| | Month 1 | 51 | 2.04 | 5.9 | 5.9 | 0.00 | 0.0 | 1.000 | — |
| | Month 2 | 47 | 1.94 | 6.4 | 17.0 | −0.11 | −5.2 | 0.227 | — |
| | Month 3 | 41 | 1.95 | 4.9 | 14.6 | −0.10 | −4.8 | 0.289 | — |
| | Month 4 | 43 | 1.91 | 4.7 | 18.6 | −0.14 | −6.8 | 0.109 | — |

^{a}Data failed normality; thus, a Wilcoxon signed rank test was used to test the null hypothesis that the mean change from baseline is zero.
— Indicates no statistically significant change in scores when compared with baseline.

| | Time | | | Subject | Subject | Mean | Mean | Signed Rank | |
|---|---|---|---|---|---|---|---|---|---|
| Treatment | Point | N | Mean | Increased, % | Decreased, % | Change | Change, % | Test | P Value^{a} |

Record the heaviness of your regular menstrual flow?
Record the heaviness of your menstrual flow this month?
1 = Light, 2 = Medium, 3 = Heavy

| | | | | | | | | | |
|---|---|---|---|---|---|---|---|---|---|
| Women's | Baseline | 55 | 2.40 | | | | | | |
| Menstrual | Month 1 | 52 | 2.25 | 9.6 | 21.2 | −0.13 | −5.6 | 0.168 | — |
| Health | Month 2 | 48 | 2.08 | 8.3 | 35.4 | −0.29 | −12.3 | 0.002 | ò |
| Powder | Month 3 | 39 | 2.10 | 5.1 | 33.3 | −0.31 | −12.8 | 0.007 | ò |
| Supplement | Month 4 | 46 | 2.13 | 8.7 | 34.8 | −0.26 | −10.9 | 0.012 | ò |
| Placebo | Baseline | 54 | 2.37 | | | | | | |
| | Month 1 | 51 | 2.08 | 3.9 | 27.5 | −0.29 | −12.4 | 0.003 | ò |
| | Month 2 | 47 | 2.11 | 4.3 | 29.8 | −0.28 | −11.6 | 0.004 | ò |
| | Month 3 | 41 | 2.12 | 4.9 | 26.8 | −0.22 | −9.4 | 0.022 | ò |
| | Month 4 | 43 | 2.16 | 4.7 | 20.9 | −0.21 | −8.8 | 0.045 | ò |

^{a}Data failed normality; thus, a Wilcoxon signed rank test was used to test the null hypothesis that the mean change from baseline is zero.
— Indicates no statistically significant change in scores when compared with baseline.
ò Indicates a statistically significant (P ≤ .05) decrease in scores when compared with baseline.

### Results:

Analysis of tolerability questionnaire showed a statistically significant (P≤0.05) no change in scores regarding the heaviness of menstrual flow at months 1, 2, 3, and 4 for cell 1 (powder supplement), and at each post-baseline time point (months 1, 2, 3, and 4) for cell 2 (placebo), when compared with baseline. There was a statistically significant (P≤0.05) increase in scores regarding the frequency of physical activity at month 1 when compared with baseline for cell 2 (placebo).

There was no statistically significant (P>0.05) change in scores regarding the cycle length, period length, cycle variance, or stress level for either cell; or in the frequency of physical activity for cell 1, at any post-baseline time point (month 1, 2, 3, or 4) when compared with baseline.

### OVERALL CONCLUSIONS

Overall results from this single-center, randomized, controlled pilot study indicate that the supplement was effective in easing PMS symptoms when used over the course of 3 months by healthy women with consistent menstrual cycles who regularly experience PMS symptoms, under the conditions of this test. After a 1-month regression period, subjects reported a statistically significant (P≤0.05) decrease in scores for appetite increase and palpitations in the week before period.

Based on the low number of product-related AEs, the supplement was well tolerated. One subject (1.8% of the 55 subjects who used the test product) experienced a nonserious AE determined to be possibly related to the test product, a figure similar to the percentage in the placebo cell (I subject or 1.9% of the 54 subjects who used the placebo). Additionally, the supplement was well perceived by the subjects.

1. A composition for preventing and/or menstrual cycle symptoms or discomforts, comprising:
(a) an ingredient or an extract of chasteberry fruit;
(b) an ingredient or an extract of saffron red sargol stigma, wherein the ingredient or the extract of saffron red sargol stigma is encapsulated by maltodextrin; and
(c) an excipient.

2. The composition of claim 1, wherein the extraction ratio of the extract of chasteberry fruit is 6:1, and wherein the extract of chasteberry fruit is extracted with a 50% ethanol extraction.

3. The composition of claim 1, wherein the extraction ratio of the extract of chasteberry fruit is 3.33:1 standardized to 2% crocins, and wherein the extract of chasteberry fruit is extracted with 30% ethanol.

4. The composition of claim 1, wherein the amount of the extract of chasteberry in the composition is in the range of from about 20 to about 40 mg.

5. The composition of claim 1, wherein the amount of the extract of chasteberry in the composition is about 30 mg.

6. The composition of claim 1, wherein the amount of the extract of saffron red sargol stigma in the composition is in the range of from about 30 to about 60 mg.

7. The composition of claim 1, further comprising at least one other botanical ingredient selected from the group consisting an ingredient or an extract of ginger, turmeric, gingko biloba, fennel, dong quai (Angelica sinensis), and pine bark.

8. The composition of claim 1, wherein the excipient comprises a disintegrant, a binder, a surfactant, an emulsifier, a viscosity modifier, a lubricant, a sweetener, a pH-adjusting agent, a preservative, a flavoring agent, a coloring agent, or an antioxidant.

9. The composition of claim 1, wherein the excipient is selected from gellan gum, carob bean gum, locust bean gum, carrageenan, alginates, agar, guar gum, xanthan gum, carboxymethyl cellulose, clear starch, pectin, gelatin, cornstarch, katakuri starch, potato starch, and gum arabic.

10. The composition of claim 1, wherein the excipient comprises at least one of:
a sweetener selected from high fructose corn syrup, mannose, maltose, glucose polymers, sucrose, glucose, dextrose, lactose, galactose, fructose, polysaccharides, rice syrup, honey, saccharin, cyclamates, acetosulfam, sorbitol, sucralose, xylitol, erythritol, Stevia extract, L-aspartyl-L-phenyl-alanine ester, L-aspartyl-D-alanine alkyl amides, L-aspartyl-L-1-hydroxymethylalkaneamide, and L-aspartyl-1-hydroxyethylalkaneamide;
a pH-adjusting agent selected from hydrochloric acid, citric acid, sodium hydrogen carbonate, potassium hydroxide, sodium hydroxide, and sodium carbonate;
a preservative selected from sorbic acid, benzoic acid, sodium benzoate, calcium benzoate, potassium benzoate, potassium sorbate, calcium sorbate, and sodium sorbate; and
a flavoring agent selected from almond oil, amaretto oil, anethole, anise oil, benzaldehyde, blackberry, black walnut oil, blueberry, caraway, caraway oil, cardamom oil, cardamom seed, cherry juice, cherry syrup, cinnamon, cinnamon oil, cinnamon water, citric acid, citric acid syrup, clove oil, cocoa, coriander oil, dextrose, eriodictyon, ethyl acetate, ethyl vanillin, fennel oil, ginger, glucose, glycerin, glycyrrhiza, grape, honey, lavender oil, lemon oil, lime, mannitol, methyl salicylate, myristica oil, orange oil, orange peel, orange syrup, peppermint, peppermint oil, peppermint water, phenylethyl alcohol, pineapple, raspberry juice, raspberry syrup, rosemary oil, rose oil, rose water, sarsaparilla syrup, sorbitol, spearmint, spearmint oil, strawberry, sucrose, thyme oil, tolu balsam, tropical, vanilla, vanillin, and wild cherry syrup.

11. The composition of claim 1, wherein the composition is a food product.

12. The composition of claim 1, wherein the composition is a dietary supplement.

13. The composition of claim 1, wherein the composition is in a form of a beverage or a gel.

14. The composition of claim 1, wherein the composition is:
a packaged beverage provided in a unit that contains between 10-1000 mL of the beverage; or
a packaged beverage provided in a unit that contains between 10-500 mL of the beverage.

15. The composition of claim 1, wherein the composition is in a form of an instant beverage.

16. The composition of claim 1, wherein the composition is a packaged gel provided in a unit that contains between 5-100 grams of the gel.

17. The composition of claim 1, wherein the composition is in a solid dose format.

18. The composition of claim 1, wherein the composition is for consumption once or more times a day.

19. The composition of claim 1, wherein the composition is for daily consumption for a minimum of 1 menstrual cycle; daily consumption for a minimum of 2 menstrual cycles; daily consumption for a minimum of 3 menstrual cycles; or a daily consumption for a minimum of 6 menstrual cycles.

20. The composition of claim 1, wherein the menstrual cycle symptoms or discomforts comprise physical, physiological and/or psychological symptoms or discomforts experienced during the monthly menstrual cycle;
wherein the physical menstrual symptoms or discomforts are bloating, abdominal discomfort, lower back pain, cramps in the pelvic area, constipation, diarrhea, swelling or tenderness in the breasts, cyclic acne, joint or muscle pain, and food cravings;
wherein the psychological symptoms or discomforts are stress, anxiety, difficulty with sleep, headache, feeling tired/fatigue, mood swings, increased emotional sensitivity, changes in interest in sex, problems with concentration and memory, and depression.

21. A capsule comprising the composition of claim 1.

22. An instant beverage comprising:
(a) an ingredient or an extract of chasteberry fruit;
(b) an ingredient or an extract of saffron red sargol stigma, wherein the ingredient or the extract of saffron red sargol stigma is encapsulated by maltodextrin; and
(c) an excipient.

23. A method of preventing and/or ameliorating menstrual cycle symptoms and/or discomforts in a subject in need thereof, comprising orally consuming an effective amount of
(a) an extract of chasteberry fruit;
(b) an extract of saffron red sargol stigma, wherein the extract of saffron red sargol stigma is encapsulated by maltodextrin,
thereby preventing and/or ameliorating the subject's menstrual cycle symptoms or discomforts.

24. The method of claim 23, wherein ingredients (a) and (b) provide prevention and/or treatment of the menstrual cycle symptoms and/or discomforts, wherein the menstrual cycle symptoms and/or discomforts comprise physical, physiological and/or psychological symptoms or discomforts experienced during the monthly menstrual cycle of the subject;
wherein the physical menstrual symptoms and/or discomforts are bloating, abdominal discomfort, lower back pain, cramps in the pelvic area, constipation, diarrhea, swelling or tenderness in the breasts, cyclic acne, joint or muscle pain, and food cravings;
wherein the psychological symptoms and/or discomforts are stress, anxiety, difficulty with sleep, headache, feeling tired/fatigue, mood swings, increased emotional sensitivity, changes in interest in sex, problems with concentration and memory, and depression.

25. The method of claim 23, wherein the ingredients (a) and (b) are orally consumed as a composition.

26. The method of claim 23, wherein:
the step of orally consuming the ingredients (a) and (b) occurs prior to, during, or after the onset of the menstrual cycle symptoms and/or discomforts;
the step of orally consuming the ingredients (a) and (b) occurs once or more times per day;
the step of orally consuming the ingredients (a) and (b) occurs daily for a minimum of 1 menstrual cycle;
the step of orally consuming the ingredients (a) and (b) occurs daily for a minimum of 2 menstrual cycles;
the step of orally consuming the ingredients (a) and (b) occurs daily for a minimum of 3 menstrual cycles; or
the step of orally consuming the ingredients (a) and (b) occurs daily for a minimum of 6 menstrual cycles.

27. The method of claim 23, wherein the subject is orally consuming:
i) between about 20 to about 40 mg per day of the extract of chasteberry fruit;
ii) between about 30 to about 60 mg per day of the extract of saffron red sargol stigma.

28. A composition for upregulating the expression of the XIST gene in a female subject comprising:
i) between about 20 to about 40 mg per day of the extract of chasteberry fruit;
ii) between about 30 to about 60 mg per day of the extract of saffron red sargol stigma.
